(19) United States
(12) Patent Application Publication
(10) Pub. No.: US 2006/0111571 A1
(43) Pub. Date: May 25, 2006

(54) Tadalafil crystal forms and processes for preparing them

(76) Inventors: Shlomit Wizel, Petah Tiqva (IL); Barnaba Krochmal, Jerusalem (IL); Ariel Givant, Jerusalem (IL); Dov Diller, Jerusalem (IL)

Correspondence Address: KENYON & KENYON LLP, ONE BROADWAY, NEW YORK, NY, 10004, US

(21) Appl. No.: 11/265,880

(22) Filed: Nov. 2, 2005

Related U.S. Application Data
(60) Provisional application No. 60/624,412, filed on Nov. 2, 2004.
(60) Provisional application No. 60/642,216, filed on Jan. 7, 2005.

(51) Int. Cl. C07D 241/36

(52) U.S. Cl. 544/343

(57) ABSTRACT

The present invention provides tadalafil crystal forms II, III, IV, VI, VII, and VIII, and processes for preparing these forms. The present invention also provides processes for preparing tadalafil forms I and V.

### RELATED APPLICATIONS

This application claims the benefit of U.S. application Ser. Nos. 60/624,412, filed Nov. 2, 2004, and 60/642,216, filed Jan. 7, 2005.

### FIELD OF THE INVENTION

The invention relates to crystalline forms of tadalafil and methods of their synthesis.

### BACKGROUND OF THE INVENTION

Tadalafil, (6R-trans)-6-(1,3-benodioxol-5-yl)-2,3,6,7,12,12a-hexahydro-2-methyl-pryazino[1,2′:1,6]pyrido[3,4b]indole-1,4-dione, is a white crystalline powder. (CAS# 171596-29-5).

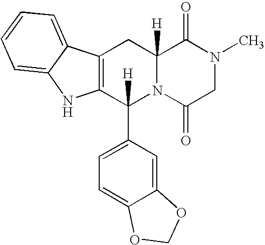

Tadalafil is currently marketed as Cialis. Cialis was developed by Eli Lilly as a treatment for impotence. In this capacity, it is reported that tadalafil functions by inhibiting the formation of cyclic guanosine monophosphate (cGMP)-specific phosphodiesterase type 5 (PDE5). The inhibition of PDE5 presumably lessens impotence by increasing the amount of cGMP, resulting in smooth muscle relaxation and increased blood flow.

Polymorphism, the occurrence of different crystal forms, is a property of some molecules and molecular complexes. A single molecule, like Tadalafil, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of tadalafil.

Repetition of the procedure described U.S. Pat. No. 5,859,006 results in crystalline anhydrous tadalafil form I (using MeOH). Crystalline anhydrous tadalafil form I is characterized by at least one of: an x-ray diffraction pattern with characteristic reflections at about 7.3°, 10.6°, 12.6°, 14.6°, 18.5°, 21.8°, and 24.3°±0.2° 2θ, or a DSC thermogram with a single endotherm at about 300° C.

Repetition of the procedure described WO 04/011463 006 results in crystalline anhydrous tadalafil form V (using Acetic acid). Crystalline anhydrous tadalafil form V is characterized by an x-ray diffraction pattern with characteristic reflections at about 8.3°, 15.1°, 18.8°, 19.2°, and 20.3°±2° 2θ. The crystalline form may be further characterized by a DSC thermogram with two endotherms at about 110° C. and at about 300° C. Tadalafil form V may be further characterized by TGA, showing a weight loss of about 13% at a temperature of between about 25° C. to about 150° C. Tadalafil form V may be further characterized by Karl-Fisher, showing water content of less than 1%. The weight loss corresponds to the theoretical value of tadalafil Acetic acid of 4:1.

### SUMMARY OF THE INVENTION

In one aspect, the present invention provides a method of preparing crystalline anhydrous tadalafil Form I by crystallizing it from a solvent selected from the group consisting of 2-methoxyethanol, absolute ethanol, acetonitrile, 1-propanol, isopropanol, ethyl acetate, toluene dimethyl sulfoxide (“DMSO”) , n-butanol, chloroform, tetrahydrofuran (“THF”) and mixtures thereof.

In yet a further aspect, the present invention provides a method of preparing crystalline anhydrous tadalafil Form I that includes the steps of dissolving tadalafil in a solvent selected from the group consisting of chloroform, methylene chloride, THF, and acetone; combining this solution with an organic anti-solvent selected from the group consisting of petroleum ether, cyclohexane, toluene, xylenes, benzene, and methyl-tert-butyl ether (“MTBE”) to obtain a precipitate; and isolating the precipitate.

In another aspect, the present invention provides an anti-solvent crystallization method of preparing crystalline anhydrous tadalafil Form I comprising the steps of dissolving tadalafil in THF; combining the solution with an anti-solvent selected from the group consisting of petroleum ether, heptane and hexane; adding anti-solvent that is methanol until a precipitate is obtained; and isolating the precipitate.

In a further aspect, the present invention provides an exhaustive drying process for preparing anhydrous crystalline tadalafil Form I that comprises dissolving crystalline tadalafil in an aliphatic ketone selected from the group consisting of methylethyl ketone, isobutyl ketone and acetone; cooling the solution to obtain a precipitate; isolating the precipitate; and exhaustively drying the tadalafil at about 45° C. to about 90° C. to obtain the crystalline form.

In yet another aspect, the present invention provides a process for preparing anhydrous crystalline tadalafil Form I by dissolving tadalafil in an aliphatic ketone selected from the group consisting of methylethyl ketone and acetone; cooling the solution to obtain a precipitate; isolating the precipitate; and exposing it to high humidity to obtain the crystalline form.

In one aspect, the present invention provides a crystalline form of tadalafil Form II characterized by an x-ray diffraction pattern with reflections at about 7.6°, 14.0°, 15.2°, 18.0°, and 22.8°±2° 2θ.

In another aspect, the present invention provides a process for preparing anhydrous crystalline tadalafil Form I comprising exposing one of the group consisting of crystalline tadalafil Form II methylethyl ketone solvate and crystalline tadalafil Form II acetone solvate to high humidity to obtain the crystalline form.

In one aspect, the present invention provides crystalline tadalafil Form III, characterized by an x-ray diffraction pattern with reflections at about 8.3°, 13.5°, 7.7°, and 18.4°±2° 2θ.

In another aspect, the present invention provides a process for preparing anhydrous crystalline tadalafil Form I comprising exposing one of the group consisting of crystalline tadalafil Form III methylethyl ketone solvate and crystalline tadalafil Form III acetone solvate to high humidity to obtain the crystalline form.

In a further aspect, the present invention provides an exhaustive drying process for preparing a mixture of tadalafil Form I and form III, comprising exhaustively drying crystalline tadalafil selected from the group consisting of crystalline tadalafil Form II methylethyl ketone solvate and crystalline tadalafil Form II at a temperature of between about 50° C. to about 75° C.

In one aspect, the present invention provides a crystalline form of tadalafil Form IV, characterized by an x-ray diffraction pattern with reflections at about 7.6°, 10.6°, 15.2°, 18.4°, and 22.7°±2° 2θ.

In yet another aspect, the present invention provides a method of preparing crystalline tadalafil Form V, including the steps of providing a solution of tadalafil in acetic acid; cooling the solution until a precipitate is obtained; and isolating the precipitate.

In a further aspect, the present invention provides a crystalline form of tadalafil Form VI, characterized by at least one of: an x-ray diffraction pattern with reflections at about 7.1°, 9.3°, 11.4°, 13.5°, 17.8°, 19.2°, 21.2° 2θ, or by an exotherm in DSC at about 200° C. and a melting endotherm at about 300° C.

In a further aspect, the present invention provides a crystalline form of tadalafil Form VII, characterized by at least one of: an x-ray diffraction pattern with reflections at about 7.0°, 13.1°, 17.6°, 19.0°, 20.9°, 24.6° 2θ, or by two endotherms in DSC: a broad endotherm at about 170° C. and a melting endotherm at about 300° C.

In another aspect, the present invention provides crystalline tadalafil Form VIII, characterized by an x-ray diffraction pattern with reflections at about 7.2°, 7.6°, 8.2°, 13.3°, 17.6°, 18.2°, 22.6°±2° 2θ.

In a further aspect, the present invention provides a method of preparing crystalline tadalafil Form VIII, by performing one of the following: heating crystalline tadalafil form IV at a temperature of between about 50° C. to about 70° C. to obtain crystalline tadalafil Form VIII; or heating crystalline tadalafil Form IV, at a temperature of between about 40° C. to about 70° C. to obtain a mixture of crystalline tadalafil forms, wherein the crystalline forms are Form VIII and Form I.

In yet another aspect, the present invention provides a method of preparing crystalline tadalafil Form I by heating crystalline tadalafil Form IV at a temperature of between about 40° C. to about 80° C. to obtain a mixture of crystalline tadalafil forms VIII and I.

### BRIEF DESCRIPTION OF THE FIGURES

FIG. 1 illustrates an x-ray diffraction diagram of anhydrous crystalline tadalafil Form I.

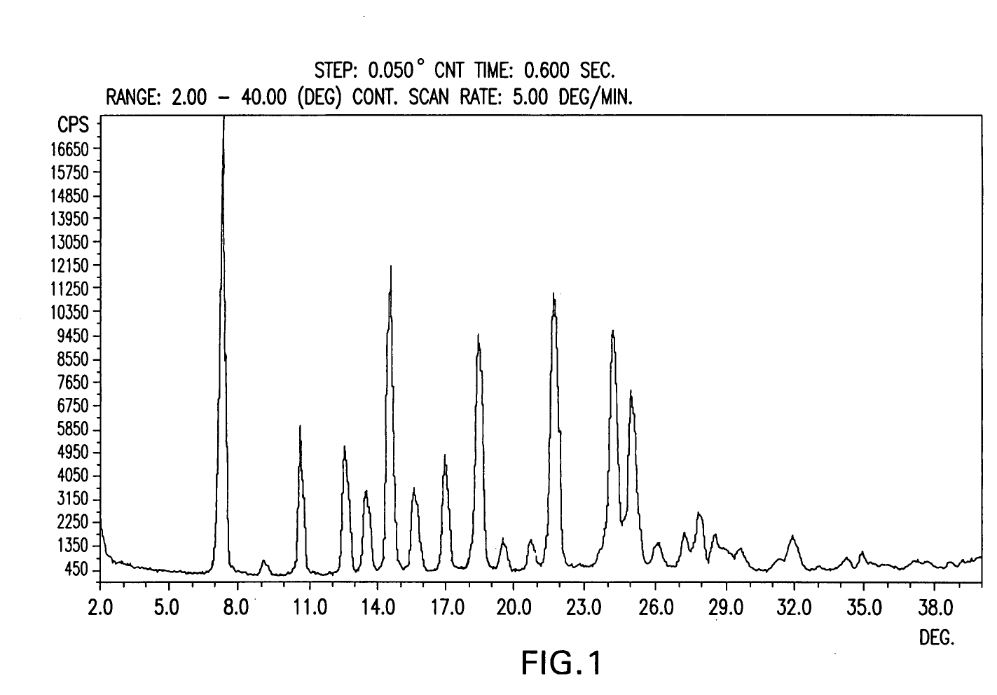

FIG. 2 illustrates an x-ray diffraction diagram of crystalline tadalafil Form II.

FIG. 3 illustrates an x-ray diffraction diagram of crystalline tadalafil Form III.

FIG. 4 illustrates an x-ray diffraction diagram of crystalline tadalafil Form IV.

FIG. 5 illustrates an x-ray diffraction diagram of crystalline tadalafil Form V.

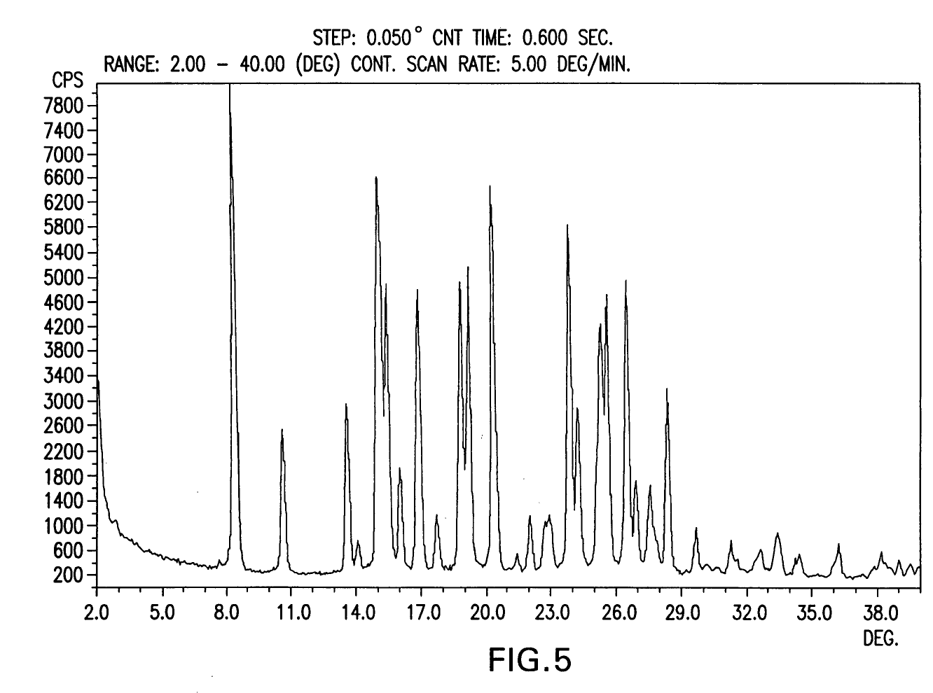

FIG. 6 illustrates an x-ray diffraction diagram of crystalline tadalafil Form VI.

FIG. 7 illustrates an x-ray diffraction diagram of crystalline tadalafil Form VII.

FIG. 8 illustrates an x-ray diffraction diagram of crystalline tadalafil Form VIII.

FIG. 9 illustrates a DSC thermogram of anhydrous crystalline tadalafil Form I.

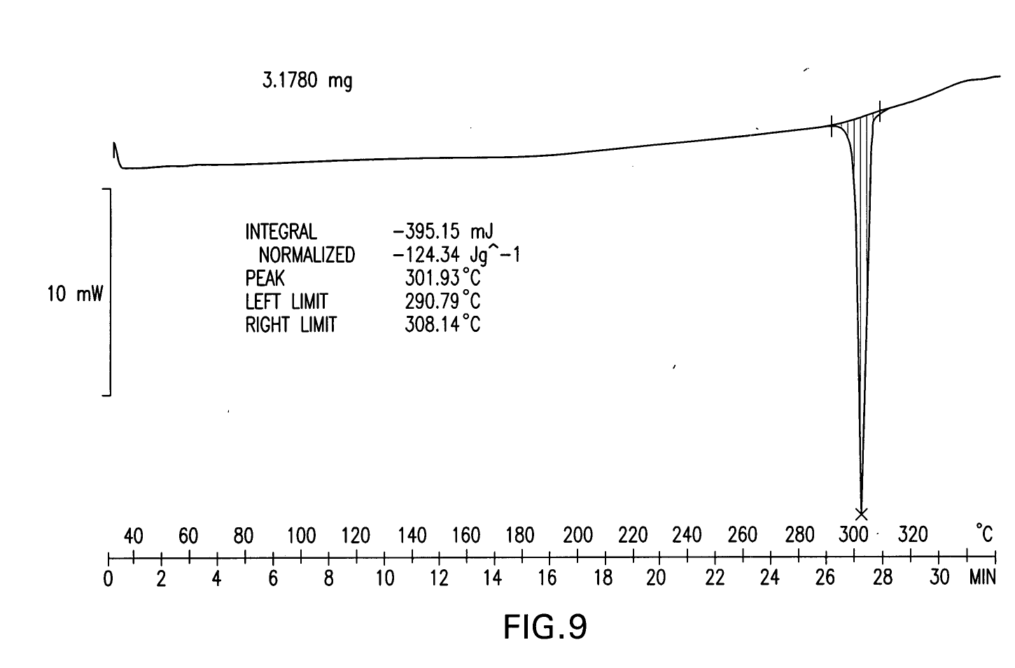

FIG. 10 illustrates a DSC thermogram of crystalline tadalafil Form II, methylethyl ketone solvate.

FIG. 11 illustrates a DSC thermogram of crystalline tadalafil Form II, acetone solvate.

FIG. 12 illustrates a DSC thermogram of crystalline tadalafil Form III, methylethyl ketone solvate.

FIG. 13 illustrates a DSC thermogram of crystalline tadalafil Form III, acetone solvate.

FIG. 14 illustrates a DSC thermogram of crystalline tadalafil Form IV.

FIG. 15 illustrates a DSC thermogram of crystalline tadalafil Form V.

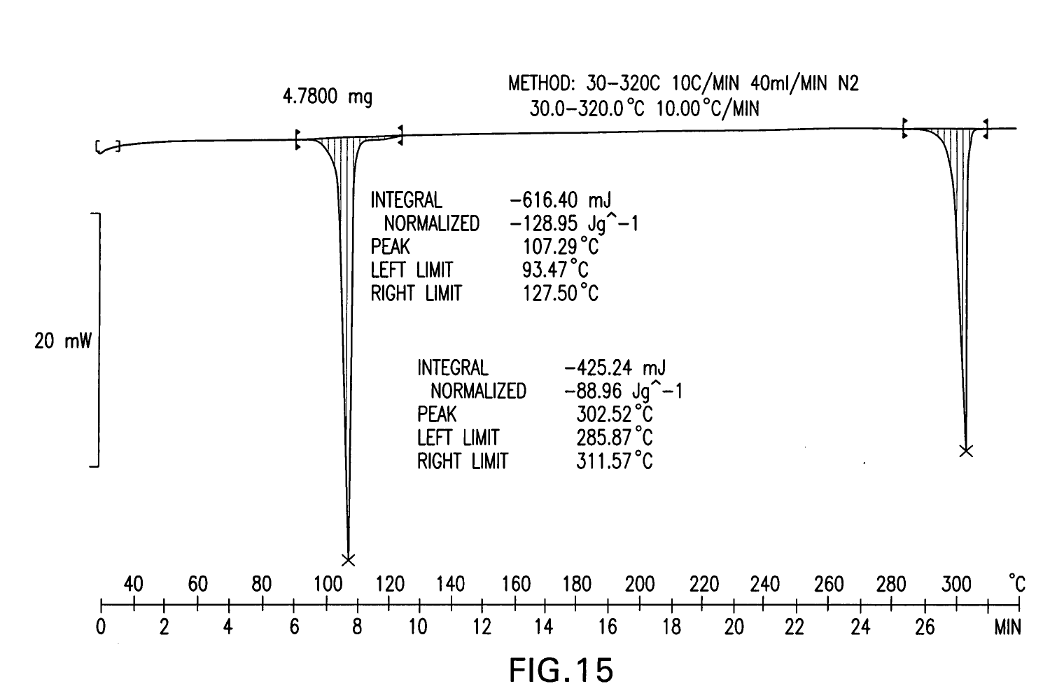

FIG. 16 illustrates a DSC thermogram of crystalline tadalafil Form VI.

FIG. 17 illustrates a DSC thermogram of crystalline tadalafil Form VII.

FIG. 18 illustrates a DSC thermogram of crystalline tadalafil Form VIII.

FIG. 19 illustrates a TGA thermogram of crystalline tadalafil Form II, methylethyl ketone solvate.

FIG. 20 illustrates a TGA thermogram of crystalline tadalafil Form II, acetone solvate.

FIG. 21 illustrates a TGA thermogram of crystalline tadalafil Form III, methylethyl ketone solvate.

FIG. 22 illustrates a TGA thermogram of crystalline tadalafil Form III, acetone solvate.

FIG. 23 illustrates a TGA thermogram of crystalline tadalafil Form IV.

FIG. 24 illustrates a TGA thermogram of crystalline tadalafil Form V.

FIG. 25 illustrates a TGA thermogram of crystalline tadalafil Form VI.

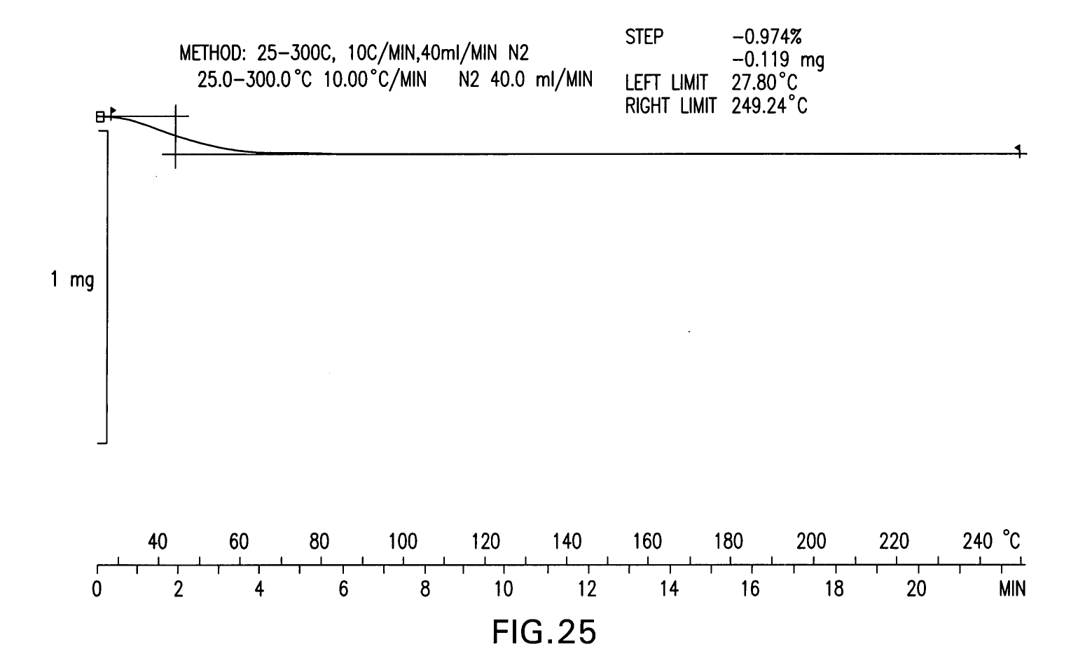

FIG. 26 illustrates a TGA thermogram of crystalline tadalafil Form VII.

FIG. 27 illustrates a TGA thermogram of crystalline tadalafil Form VIII.

### DETAILED DESCRIPTION OF THE INVENTION

The invention provides novel crystalline forms of (6R-trans)-6-(1,3-benzodioxol-5-yl)-2,3,6,7,12,12a-hexahydro-2-methyl-pryazino[1,2′:1,6]pyrido[3,4b]indole-1,4-dione. The novel crystalline forms of tadalafil have been designated Forms II, III, IV, VI, VII, and VIII. The present invention further provides methods of making each crystalline form and methods of making crystalline forms I and V of tadalafil.

By the crystallization processes of this invention, each of the novel crystal forms of tadalafil can be obtained substantially free from other crystal forms. This invention also provides crystallization processes, especially in the case of Form III, with respect to Forms II and III, and Forms I and III, in which a mixture of two forms can be obtained in the crystallization.

The term “anti-solvent” means a liquid that, when added to a solution of tadalafil in a solvent, induces precipitation of tadalafil. Precipitation of tadalafil is induced by the anti-solvent when addition of the anti-solvent causes tadalafil to precipitate from the solution more rapidly or to a greater extent than tadalafil precipitates from a solution containing an equal concentration of tadalafil in the same solvent when the solution is maintained under the same conditions for the same period of time but without adding the anti-solvent. In other words, the solubility, at a particular temperature, of tadalafil in the combination of solvent and anti-solvent is less than that in the solvent alone. Precipitation can be perceived visually as a clouding of the solution or formation of distinct particles of tadalafil suspended in or on the surface of the solution, or collected on the walls or at the bottom of the vessel containing the solution.

The term “lower alcohol” refers to an alcohol containing three or less carbons.

The term “% final volume” as used in reference to anti-solvents means the liquid volume of the anti-solvent added to the tadalafil solution as compared to the total liquid volume of the solvent and anti-solvent, or in the case of the addition of a first and second anti-solvent, the volume of the second anti-solvent as compared with the total liquid volume of solvent, first anti-solvent, and second anti-solvent.

The term “room temperature” refers to ambient temperature of from about 10° C. to about 30° C., preferably from about 18° C. to about 28° C., more preferably from about 20° C. to about 25° C., most preferably from about 21° C. to about 23° C.

The term “hot” refers to a temperature of at least above 20° C. from the starting temperature of the reaction mixture.

As used herein, the term “exhaustively dry” as used in describing processes for obtaining crystalline tadalafil Form I, refers to drying a sample of crystalline tadalafil for a time sufficient to effect conversion of the crystalline form to Form I, typically at least about 3 hours and preferably for about 24 hours.

As used herein, the terms “exhaustively drying” and “exhaustively dried” refer to both a process for obtaining crystalline tadalafil Form I, by exhaustively drying previously obtained crystalline tadalafil either Form II or Form III, and also to an additional step in the process for obtaining crystalline tadalafil Form II or Form III, which can be performed to obtain crystalline tadalafil Form I.

As used herein, the term “slurry” refers to a thin mixture of a liquid and a finely divided substance, such as any form of crystalline tadalafil.

As used herein, the term “high humidity” refers to an atmosphere in which the relative humidity is no less than about 80% and is preferably about 100%.

As used herein, the term “relative humidity” refers to the ratio of the amount of water vapor in the air at a specific temperature to the maximum amount that the air could hold at that temperature, expressed as a percentage.

As used herein, the term “aliphatic ketone” refers to an organic chemical compound having the general structure R₁C(O)R₂ wherein R₁ and R₂ are, independently, linear or branched alkyl groups having from one to four carbon atoms.

As used herein, the term “exposure time” refers to a period of time sufficient to effect conversion of crystalline tadalafil to crystalline tadalafil Form I. The exposure time is typically a period of at least about three days, and preferably a period of about seven days.

As used herein, the term “substantially free of”, in reference to the crystalline forms II, III, IV, VI, VII and VIII refers to crystalline forms having a purity of above 95%, preferably above 99%.

In one embodiment of the invention, crystalline anhydrous tadalafil Form I can be prepared by a single solvent crystallization method, or by an anti-solvent crystallization method. In the first step of each method, a solution of tadalafil is provided. The tadalafil solution can be provided by any convenient means, for example, by adding tadalafil with solvent to a suitable vessel, as determined by one skilled in the art, and heating. Possible methods of heating include sand baths, oil baths, and preferably water baths.

In one embodiment, the present invention provides a method of preparing crystalline anhydrous tadalafil Form I by crystallizing it from a solvent selected from the group consisting of 2-methoxyethanol, absolute ethanol, acetonitrile, 1-propanol, isopropanol, ethyl acetate, toluene containing dimethyl sulfoxide (“DMSO”), n-butanol, chloroform, tetrahydrofuran (“THF”) and mixtures thereof.

In another embodiment, the present invention provides a single solvent crystallization method for obtaining crystalline anhydrous tadalafil Form I, comprising the steps of dissolving tadalafil in a solvent selected from the group consisting of absolute ethanol, 1-propanol, isopropanol, 2-methoxyethanol, acetonitrile and mixtures thereof, at a temperature of from about 60° C. to about 120° C.; cooling the solution until a precipitate is obtained; and isolating the precipitate. Preferably, the tadalafil is dissolved at a temperature of about 83° C. Preferably, the solution is cooled to a temperature of below about 30° C. and above about 10° C., more preferably to about room temperature.

Depending on the concentration of the provided solution and the crystallization temperature of the solution, a holding time, or crystallization time, which is the time in which the precipitate is obtained, can be employed. Preferably, the holding time is for about 24 hours or more. Cooling can optionally be performed in steps, for example, cooling the tadalafil solution to room temperature and later cooling further to about 10° C. Suitable methods of isolation of the precipitate include centrifugation and decanting and preferably filtration.

In another embodiment, the present invention provides a single solvent crystallization method for obtaining crystalline anhydrous tadalafil Form I comprising the steps of dissolving tadalafil in a solvent selected from the group consisting of ethyl acetate, toluene containing about DMSO, n-butanol, methanol, chloroform, THF and mixtures thereof; cooling the solution until a precipitate is obtained; and isolating the precipitate. Preferably, when the solvent is DMSO, its concentration is of about 0.5% to about 5% by volume. Preferably, the tadalafil is dissolved at reflux temperature. Preferably, the solution is cooled to a temperature of between about 0° C. to about room temperature. Cooling can optionally be performed in steps, for example, cooling the solution to about room temperature and later further cooling it to about 0° C. in an ice bath for about 1 hour to complete precipitation.

In another embodiment, the present invention provides a process for preparing anhydrous tadalafil Form I by an anti-solvent crystallization method. The method comprises dissolving tadalafil in an organic solvent selected from the group consisting of chloroform, methylene chloride, THF, and acetone; combining the solution with an organic anti-solvent selected from the group consisting of petroleum ether, cyclohexane, toluene, xylenes, benzene, hexane, heptane, octane and MTBE, to obtain a precipitate; and isolating the precipitate. Preferable solvent-anti-solvent combinations include chloroform and one of the groups consisting of petroleum ether, preferably at about 40% final volume, toluene, preferably at about 73% final volume, xylenes, preferably at about 70% final volume or benzene, preferably at about 70% final volume. Additional preferable combinations of solvent and anti-solvent include methylene chloride and cyclohexane, preferably at about 40% final volume, and hot acetone and MTBE, preferably at about 50% final volume.

In another embodiment, the present invention provides a process for preparing crystalline anhydrous tadalafil Form I by an anti-solvent crystallization method using a combination of first and second anti-solvents. The process comprises dissolving tadalafil in THF; combining the solution with an anti-solvent selected from the group consisting of petroleum ether, heptane and hexane; adding an anti-solvent that is methanol until obtaining a precipitate; and isolating the precipitate. Preferably, the isolation is by filtration. Preferably, after the combination the petroleum ether is about 96% volume. Preferably, the final volume of methanol is about 23%.

In another embodiment, crystalline anhydrous tadalafil Form I, can be produced by an exhaustive drying method. The method comprises dissolving tadalafil in an aliphatic ketone selected from the group consisting of methylethyl ketone, isobutyl ketone or acetone; cooling the solution to obtain a precipitate; isolating the precipitate; and exhaustively drying it at a temperature of about 45° C. to about 90° C. to obtain the crystalline form. Preferably, the solution is cooled to about room temperature, and can optionally be further cooled to a temperature not less than about 10° C., to complete precipitation. Preferably, the precipitated crystalline tadalafil is isolated by filtration. Preferably, the drying occurs at a temperature of about 65° C. Preferably, the drying is for about 24 hours. Preferably, the drying is under atmospheric pressure.

In yet another embodiment, crystalline anhydrous tadalafil Form I can be produced in a high humidity atmosphere. The process comprises providing a solution of tadalafil in an aliphatic ketone selected from the group consisting of methylethyl ketone and acetone; cooling the solution to obtain a precipitate; isolating the precipitate; and exposing the precipitate to high humidity to obtain the crystalline form. Preferably, the solution is cooled to about room temperature, and can optionally be further cooled to a temperature not less than about 10° C., to complete precipitation. Preferably, the precipitated crystalline tadalafil is isolated by filtration. Preferably, the high humidity is a relative humidity greater than about 80%, more preferably a relative humidity of about 100%. Preferably, the precipitate is exposed to high humidity at about room temperature.

In another embodiment, the present invention provides novel crystalline forms of tadalafil which can exist as ketonates. Crystalline tadalafil Form II and crystalline tadalafil Form III can exist as ketonates, wherein the ketone can be, for example, acetone or methylethyl ketone.

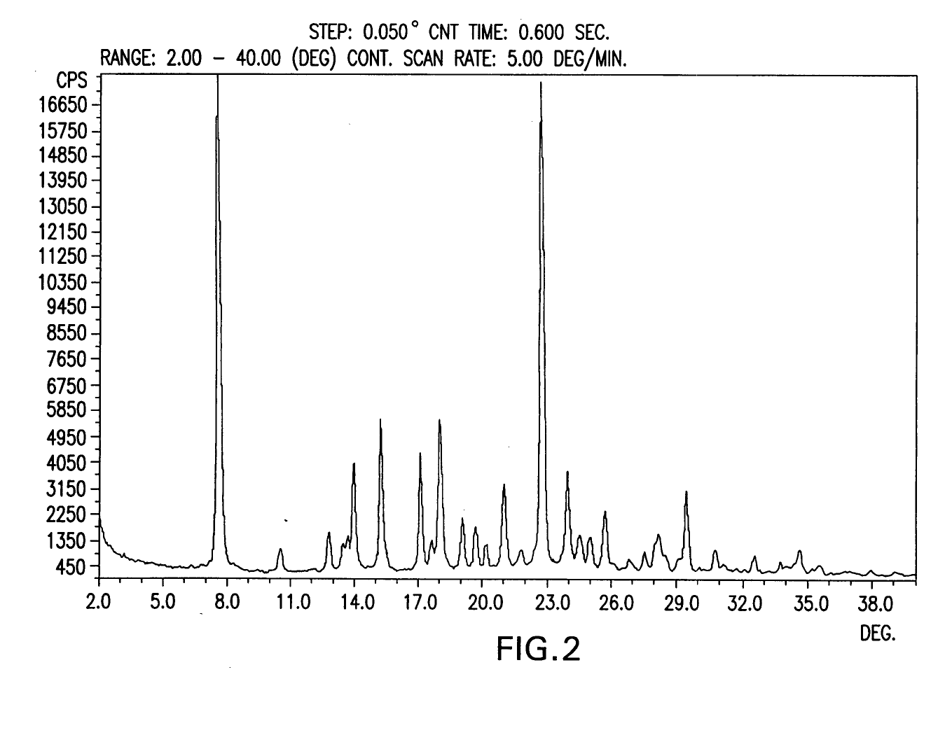

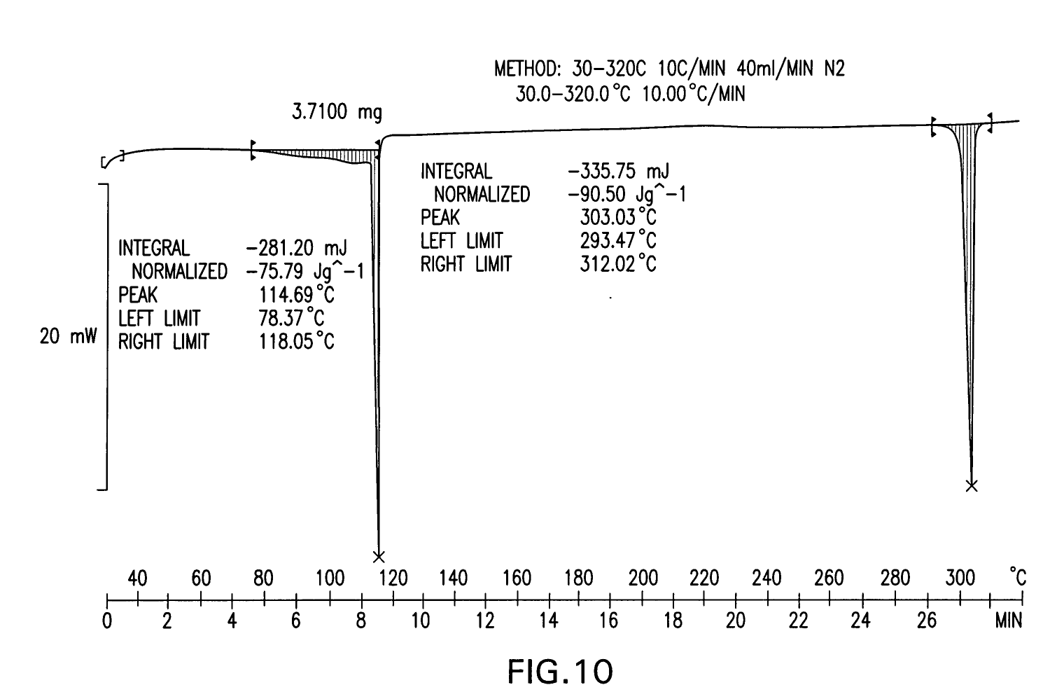

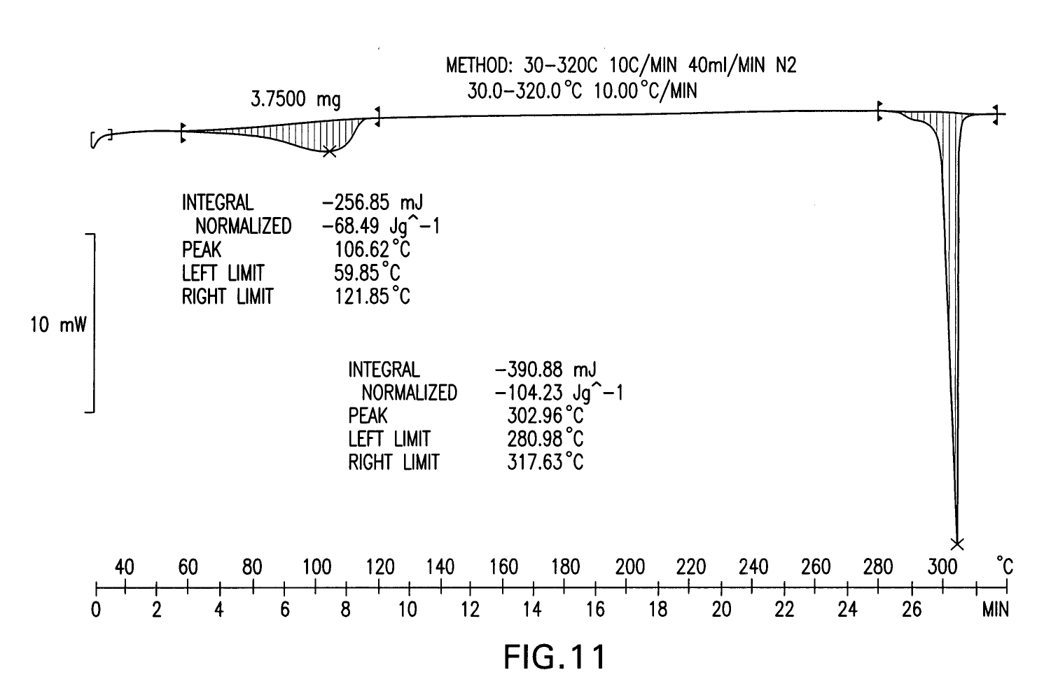

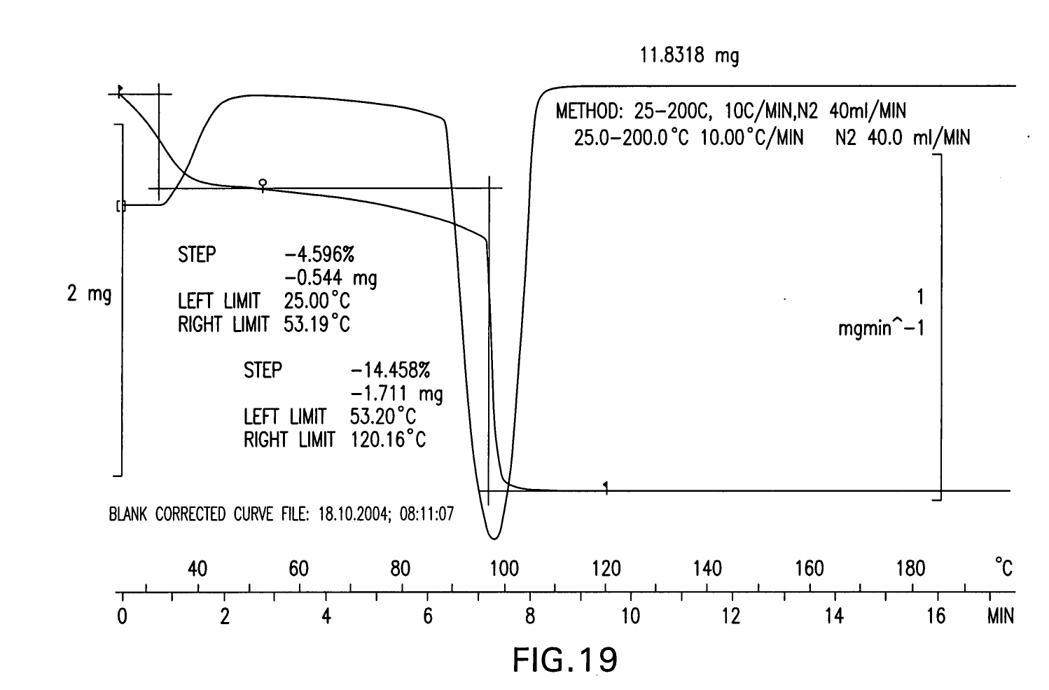

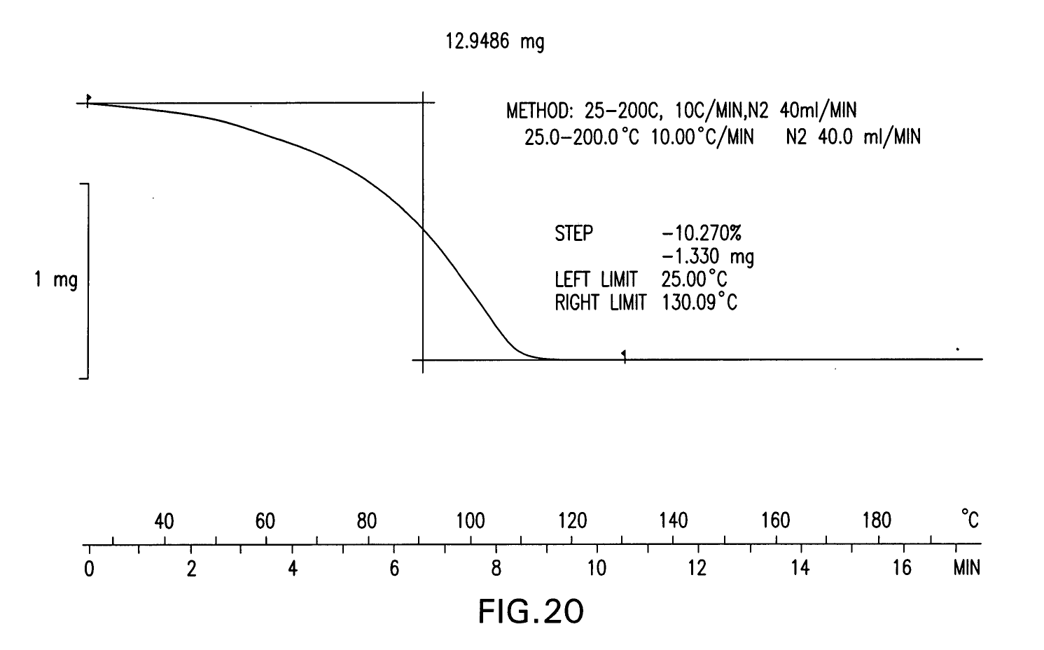

In another embodiment, the present invention provides a novel crystalline form of tadalafil Form II characterized by an x-ray diffraction pattern with characteristic reflections at about 7.6°, 14.0°, 15.2°, 18.0°, and 22.8°±2° 2θ. The crystalline form may be a ketone solvate. The ketone solvate may be methylethyl ketone solvate or acetone solvate. The crystalline form may be further characterized by two endotherms in DSC at about 105-115° C. and at about 300° C. Tadalafil form II Methylethyl ketone solvate may be further characterized by TGA, showing a weight loss of about 15-16% at a temperature of about 100° C. Tadalafil form II Methylethyl ketone solvate may be further characterized by Karl-Fisher, showing water content of less than 1%. Tadalafil form II acetone solvate may be further characterized by TGA, showing a weight loss of about 10-15% at a temperature of below about 120° C. The weight losses correspond to the theoretical value of tadalafil Methylethyl ketone solvate and tadalafil acetone solvate of 1:1 ratio. FIG. 2 depicts a characteristic x-ray diffraction pattern of crystalline tadalafil Form II. The x-ray diffraction diagram of Form II is insensitive to the identity of the ketone forming the ketone solvate. FIGS. 10 and 19 depict DSC and TGA thermograms corresponding to crystalline tadalafil Form II methylethyl ketone solvate, respectively. FIGS. 11 and 20 depict a DSC and TGA thermograms corresponding to crystalline tadalafil Form II acetone ketone solvate, respectively.

In another embodiment, the present invention provides a process for preparing crystalline tadalafil Form II by a single solvent crystallization method. The method comprises dissolving tadalafil in a ketone solvent selected from the group consisting of methylethyl ketone or acetone at a temperature of about 45° C. to about 83° C.; cooling the solution until a precipitate is obtained; and isolating the precipitate. Preferably, the tadalafil is dissolved at a temperature of about 83° C. Preferably, the solution is cooled to a temperature of between about 0° C. to about 25° C., more preferably to a temperature of about 10° C.

A holding time can be employed during the cooling. Depending on the concentration of the provided solution and the crystallization temperature of the solution, a holding time, or crystallization time, which is the time in which the precipitate is obtained, can be employed. Preferably, the holding time is for about 24 hours or more. Cooling can optionally be performed in steps, for example, cooling the tadalafil solution to room temperature and later cooling further to about 10° C.

In another embodiment, the present invention provides a process for preparing crystalline tadalafil Form II by using an anti-solvent crystallization method. The method comprises dissolving tadalafil in methylethyl ketone, combining the solution with an anti-solvent selected from the group consisting of petroleum ether, cyclohexane, or MTBE, until a precipitate is obtained, and isolating the precipitate. Preferably, the anti-solvent comprises about 50% of the final volume.

Tadalafil form I can be also obtained by drying crystalline Tadalafil form II ketone solvate at a temperature of between about 40° C. to about 90° C. Preferably, the drying is at a temperature of about 50° C. Preferably, the drying is for at least 2 days. Preferably, the drying is under atmospheric pressure

In yet another embodiment, crystalline tadalafil Form II, methylethyl ketone solvate or crystalline tadalafil Form II, acetone solvate, can be used to produce crystalline anhydrous tadalafil Form I in a high humidity atmosphere. The method comprises exposing crystalline tadalafil selected from the group consisting of crystalline tadalafil Form II, methylethyl ketone solvate and crystalline tadalafil Form II, acetone solvate to high humidity until obtaining the crystalline form. Preferably, the high humidity is a relative humidity greater than about 80%, most preferably a relative humidity of about 100%. Preferably, the exposure is at about room temperature.

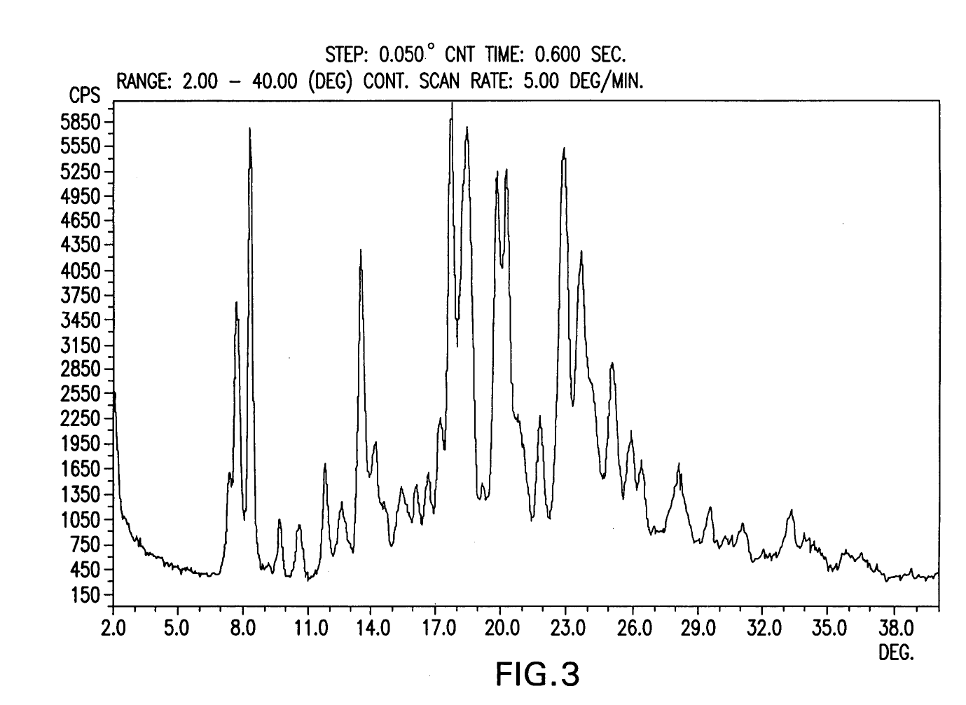

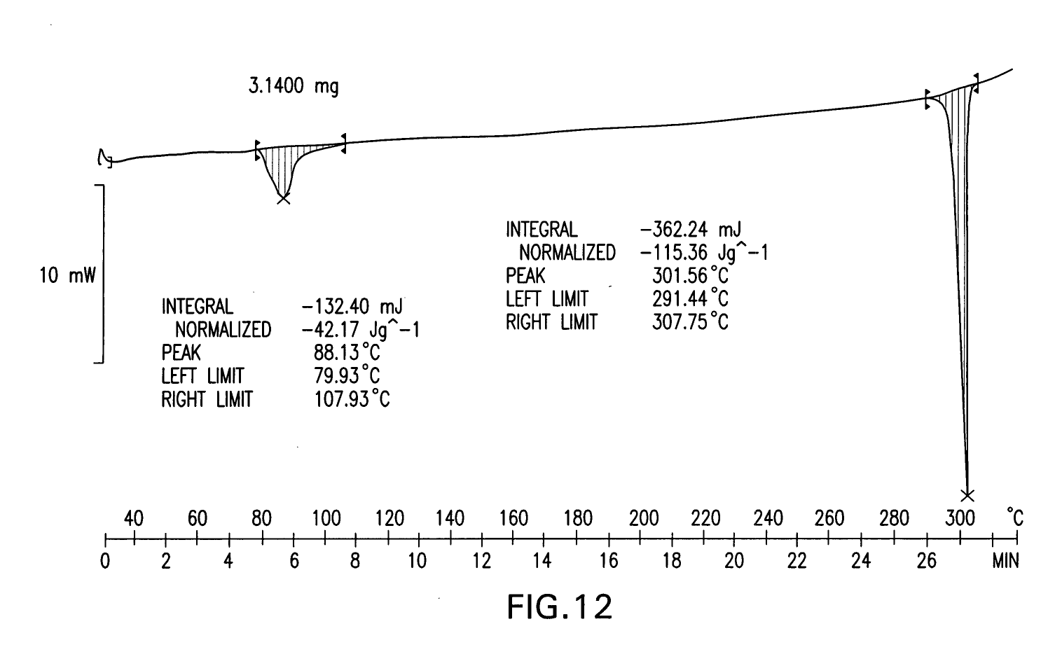

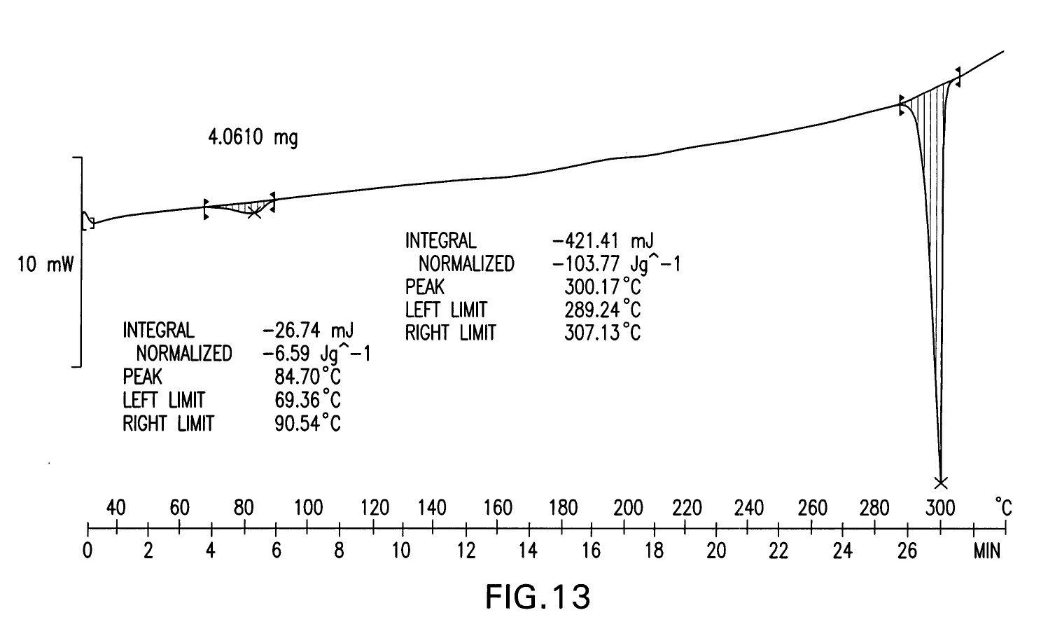

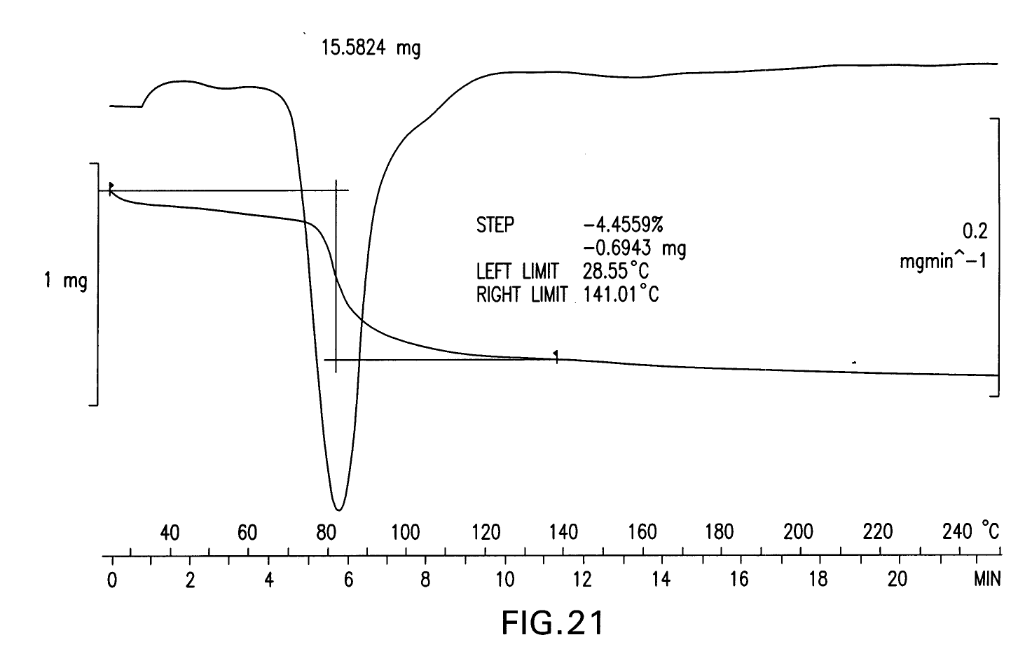

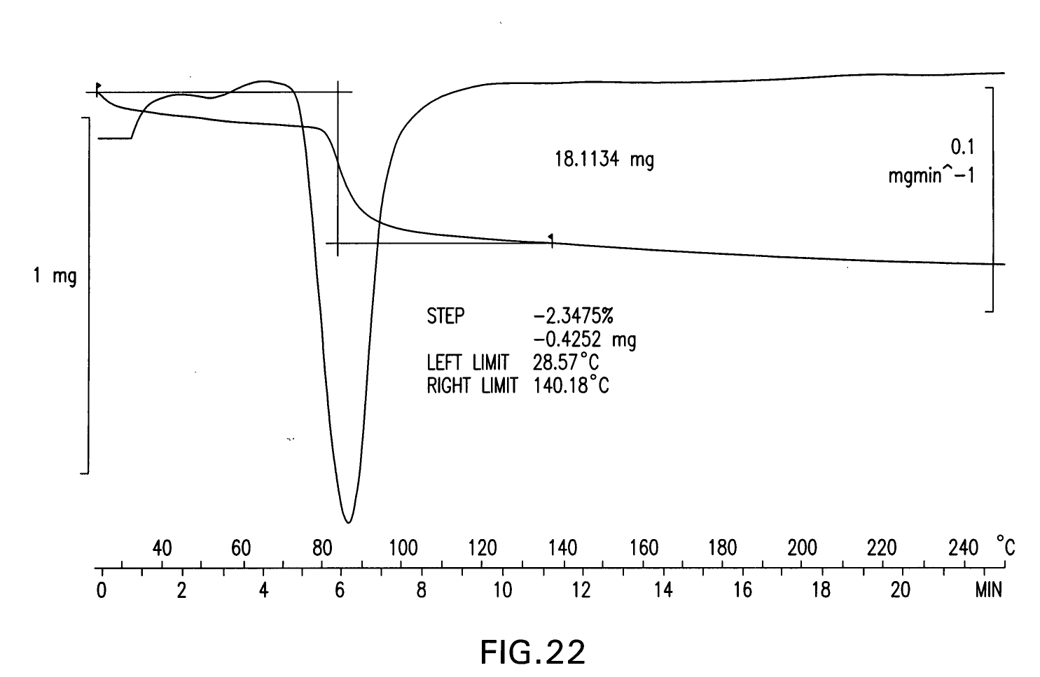

In another embodiment, the present invention provides a novel crystalline form of tadalafil Form III, ketone solvate characterized by an x-ray diffraction pattern with characteristic reflections at about 8.3°, 13.5°, 7.7°, and 18.4°±2° 2θ. The crystalline form may be further characterized by two endotherms in DSC at about 80-90° C. and at about 300° C. The crystalline form may be a ketone solvate. The ketone solvate may be methylethyl ketone solvate or acetone solvate. Tadalafil form III Methylethyl ketone may be further characterized by TGA, showing a weight loss of about 4-5% at a temperature of about 80° C. Tadalafil form III Methylethyl ketone solvate may be further characterized by Karl-Fisher, showing water content of less than 1%. The weight loss corresponds to the theoretical value of tadalafil Methylethyl ketone solvate of 4:1 ratio. Tadalafil form III acetone solvate may be further characterized by TGA, showing a weight loss of about 2-3% at a temperature of between about 25° C. to about 140° C. Tadalafil form III acetone solvate may be further characterized by Karl-Fisher, showing water content of less than 1%. The weight loss corresponds to the theoretical value of tadalafil aceone solvate of 5:1 ratio. FIG. 3 depicts a representative x-ray diffraction pattern of crystalline tadalafil Form III. The x-ray diffraction diagram of Form III is insensitive to the identity of the ketone forming the ketone solvate. FIGS. 12 and 21 depict representative DSC and TGA thermograms of crystalline tadalafil Form III methylethyl ketone solvate, respectively. FIGS. 13 and 22 depict representative DSC and TGA thermograms of crystalline tadalafil Form III, acetone solvate, respectively.

In another embodiment, the present invention provides a process for preparing a mixture of tadalafil Form II and Form III, ketone solvates by drying crystalline tadalafil Form II, ketone solvate at a temperature of about 50° C. to about 80° C. for about 0.5 to about 6 hours. Preferably, the crystalline form is dried under vacuum. Preferably, the crystalline form is dried to a temperature of about 65° C. Preferably, the crystalline form is dried for about 3 hours. A mixture of tadalafil Form II and Form III methylethyl ketone solvate, can also be obtained by drying tadalafil Form II, methylethyl ketone solvate at a temperature of about 45° C. to about 70° C. for about 0.5 to about 5 hours. Preferably, the crystalline form is dried at a temperature of about 65° C. Preferably, the crystalline form is dried under vacuum. Preferably, the crystalline form is dried for about 2 hours.

In a preferred embodiment, crystalline tadalafil Form III, acetone solvate can be obtained by drying crystalline tadalafil Form II, acetone solvate at a temperature of about 45° C. to about 70° C. for about 0.5 to about 5 hours. Preferably, the crystalline form is dried to a temperature of about 65° C. Preferably, the crystalline form is dried under vacuum. Preferably, the crystalline form is dried for about 3 hours.

Drying of Tadalafil form II ketone solvate should be controlled properly in order to get the desired crystal form. Drying of Tadalafil Ketone solvate form II by vacuum will give form III, while drying under atmospheric pressure will resulted in the formation of form I. Time of drying should be sufficient for complete conversion to the desired crystal form.

In yet another embodiment, crystalline tadalafil Form III, methylethyl ketone solvate or crystalline tadalafil Form III, acetone solvate can be used to produce crystalline anhydrous tadalafil Form I in a high humidity atmosphere. The method comprises exposing crystalline tadalafil selected from the group consisting of crystalline tadalafil Form III, methylethyl ketone solvate and crystalline tadalafil Form III, acetone solvate to high humidity. Preferably, the high humidity is a relative humidity greater than about 80%, most preferably a relative humidity of about 100%. Preferably, the exposure is at about room temperature.

In another embodiment, crystalline tadalafil Form II, methylethyl ketone solvate or crystalline tadalafil Form II, acetone solvate can be used to produce a mixture of crystalline anhydrous tadalafil Form I and crystalline tadalafil form III by an exhaustive drying method. The method comprises drying the crystalline tadalafil selected from the group consisting of crystalline tadalafil Form II, methylethyl ketone solvate and crystalline tadalafil Form II, acetone solvate, at a temperature of between about 50° C. to about 75° C. Preferably, the drying is under atmospheric pressure Preferably, the drying is at a temperature of about 65° C. Preferably, the drying is for at least about 24 hours.

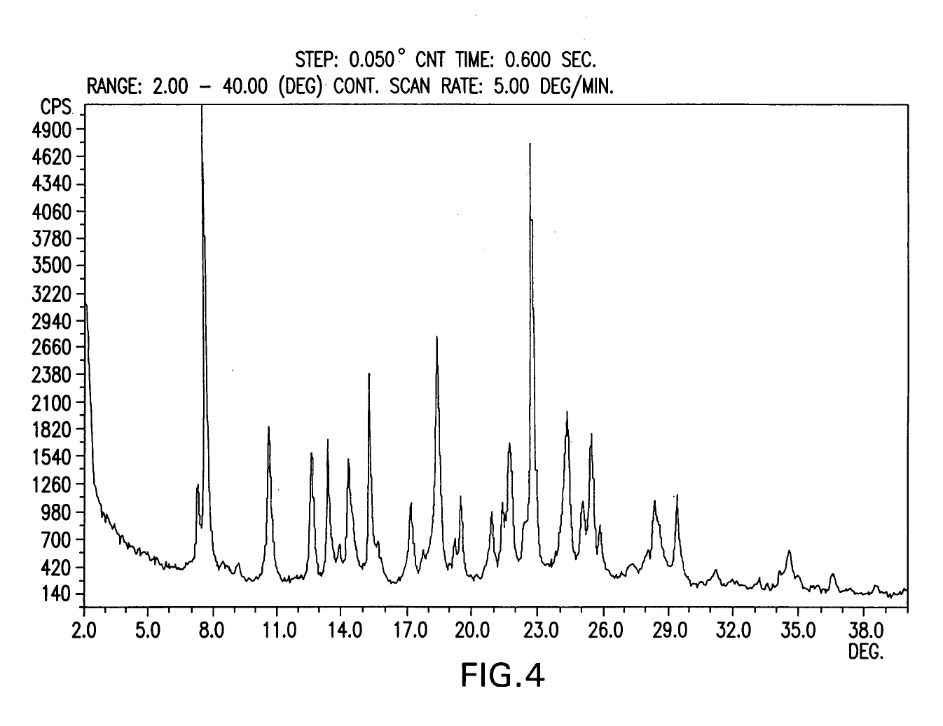

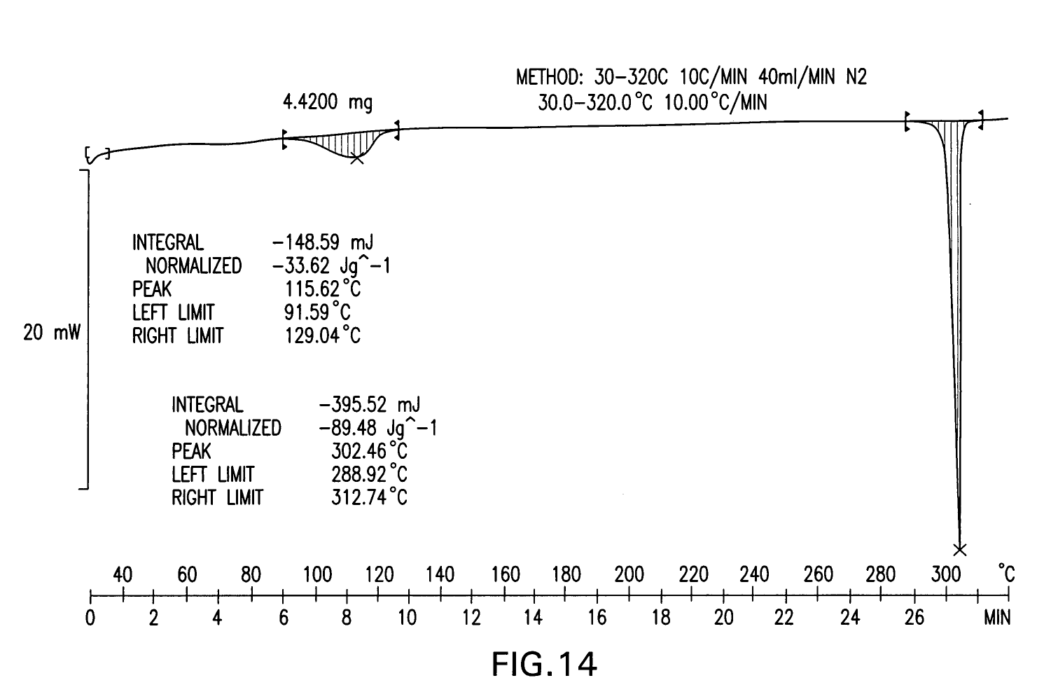

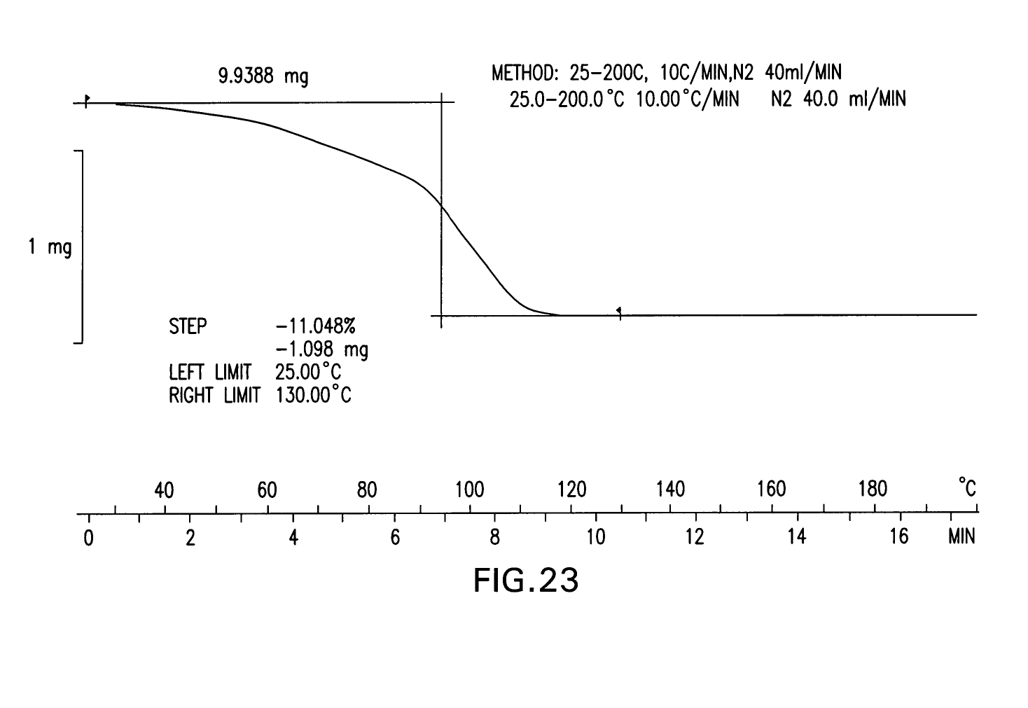

In another embodiment, the present invention provides a novel crystal form of tadalafil Form IV, characterized by an x-ray diffraction pattern with characteristic reflections at about 7.6°, 10.6°, 15.2°, 18.4°, and 22.7°±2° 2θ. The crystalline form may be further characterized by two endotherms in DSC at about 110-115° C. and at about 300° C. Tadalafil form IV may be further characterized by TGA, showing a weight loss of about 11-16% at a temperature of between about 25° C. to about 130° C. FIG. 4 depicts a characteristic x-ray diffraction pattern of crystalline tadalafil Form IV. FIGS. 14 and 23 depict characteristic DSC and TGA thermograms of crystalline tadalafil Form IV, respectively.

In another embodiment, the present invention provides a process for preparing crystalline tadalafil Form IV by a single solvent crystallization method, comprising the steps of dissolving tadalafil in methylene chloride; cooling the solution until a precipitate is obtained; and isolating the precipitate. Preferably, the dissolving step is at about reflux temperature. Preferably, the solution is cooled to a temperature of between about 0° C. to about room temperature. Preferably, the solution is first cooled to about room temperature and then is cooled to about 0° C. in an ice bath for about 1 hour to complete precipitation.

In another embodiment, the present invention provides a process for preparing crystalline tadalafil Form IV, by an anti-solvent crystallization process. The process comprises providing a solution of tadalafil in methylene chloride; combining the solution with an anti-solvent that is petroleum ether until a precipitate is formed; and isolating the precipitate. Preferably, the dissolving step is at about reflux temperature. Preferably, the petroleum ether is about 30% from the final volume.

Tadalafil form II, form III, and form IV are all characterized by an endothermic peak by DSC at about 80-120° C., and by a melting endotherm at about 300° C.

In another embodiment, the present invention provides a process for preparing crystalline tadalafil Form V by a single solvent crystallization method. The process comprises dissolving tadalafil in acetic acid; cooling the solution until a precipitate is obtained; and isolating the precipitate. Preferably, tadalafil is dissolved at about reflux temperature. Preferably, the cooling is to a temperature of between about room temperature to about 0° C. Preferably, the solution is first cooled to about room temperature and then is cooled to about 0° C. in an ice bath for about 1 hour to complete precipitation.

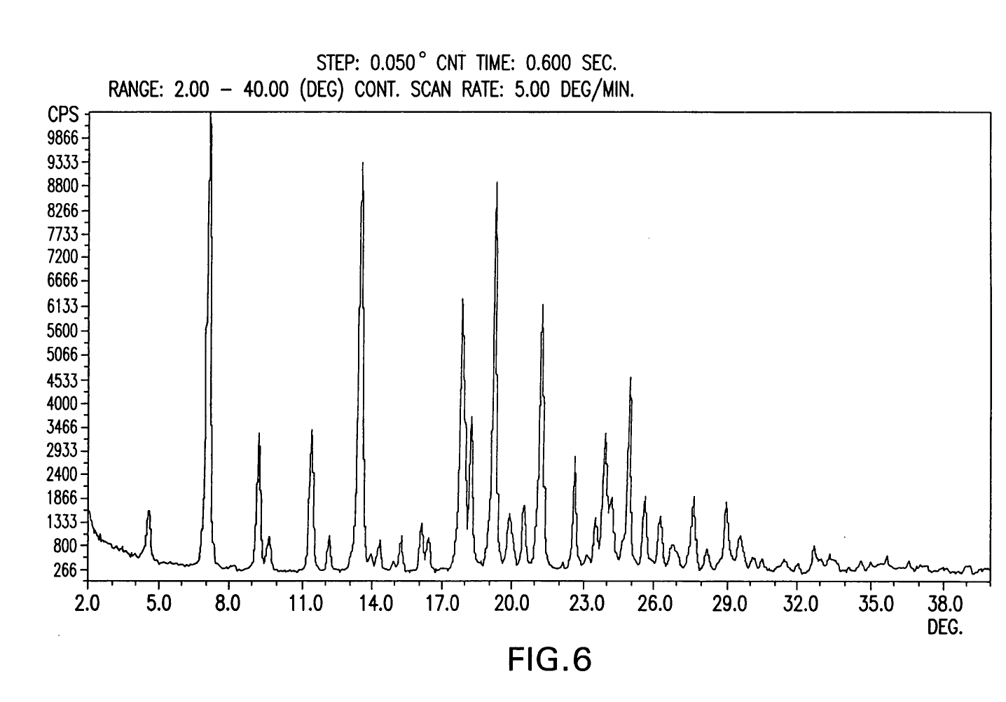

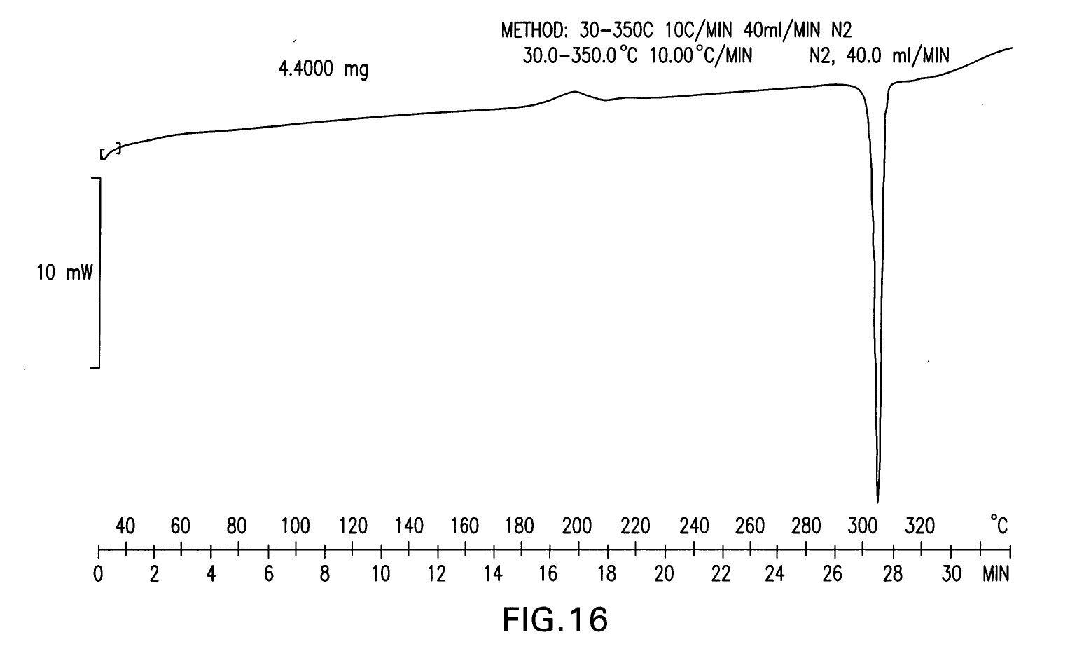

In another embodiment, the present invention provides a crystalline anhydrous form of tadalafil Form VI, characterized by at least one of: an x-ray diffraction pattern with reflections at about 7.1°, 9.3°, 11.4°, 13.5°, 17.8°, 19.2°, 21.2° 2θ, or by an exotherm in DSC at about 200° C. and a melting endotherm at about 300° C. Tadalafil form VI may be further characterized by TGA, showing a weight loss of less than 1%. FIG. 6 depicts a representative x-ray diffraction pattern of crystalline tadalafil Form VI. FIGS. 16 and 28 depict representative DSC and TGA thermograms of crystalline tadalafil Form VI, respectively.

In another embodiment, the present invention provides a process for preparing Crystalline anhydrous tadalafil Form VI by using a single solvent method. The method comprises slurrying methanol and tadalafil Form IV until obtaining a precipitate; and isolating the precipitate. Preferably, the tadalafil is isolated by filtration. Preferably, the isolated precipitate is dried at a temperature of about 40° C. to about 70° C., more preferably at a temperature of about 65° C., under vacuum, for about 3 hours.

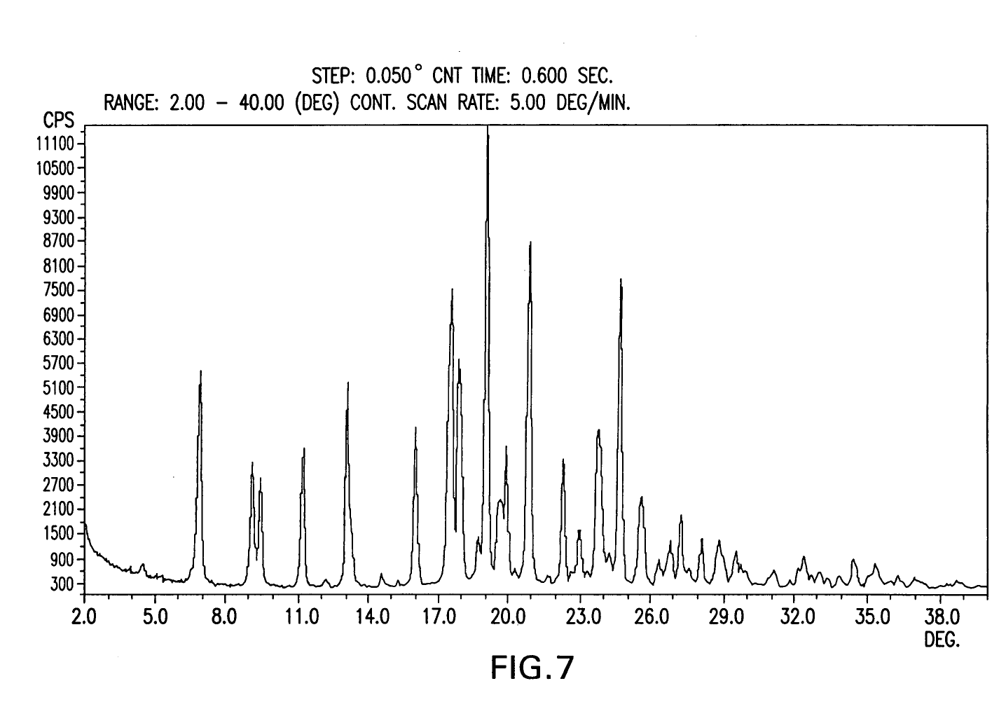

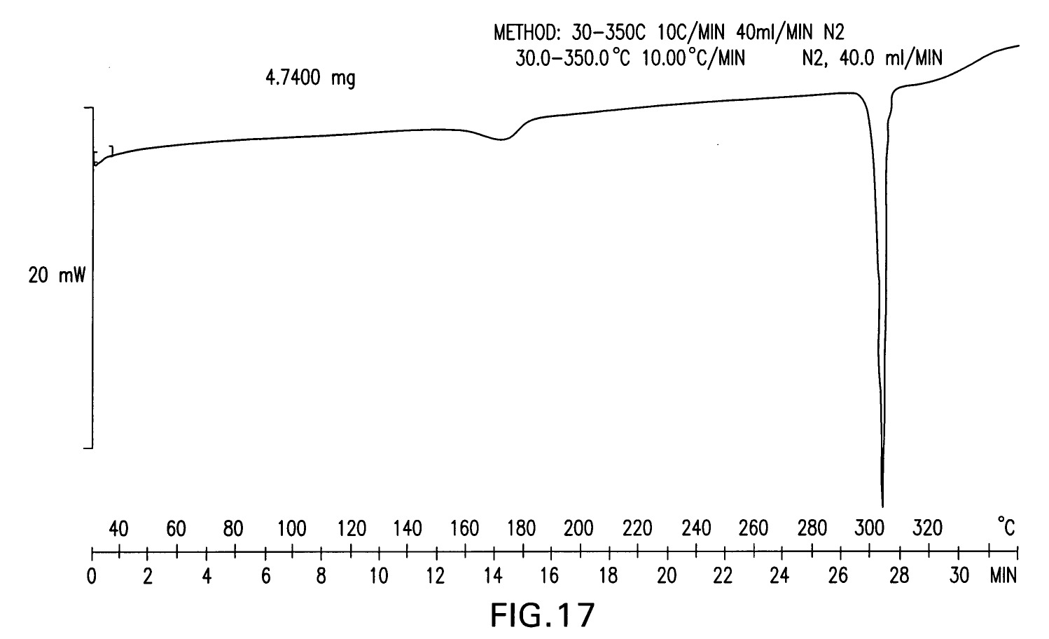

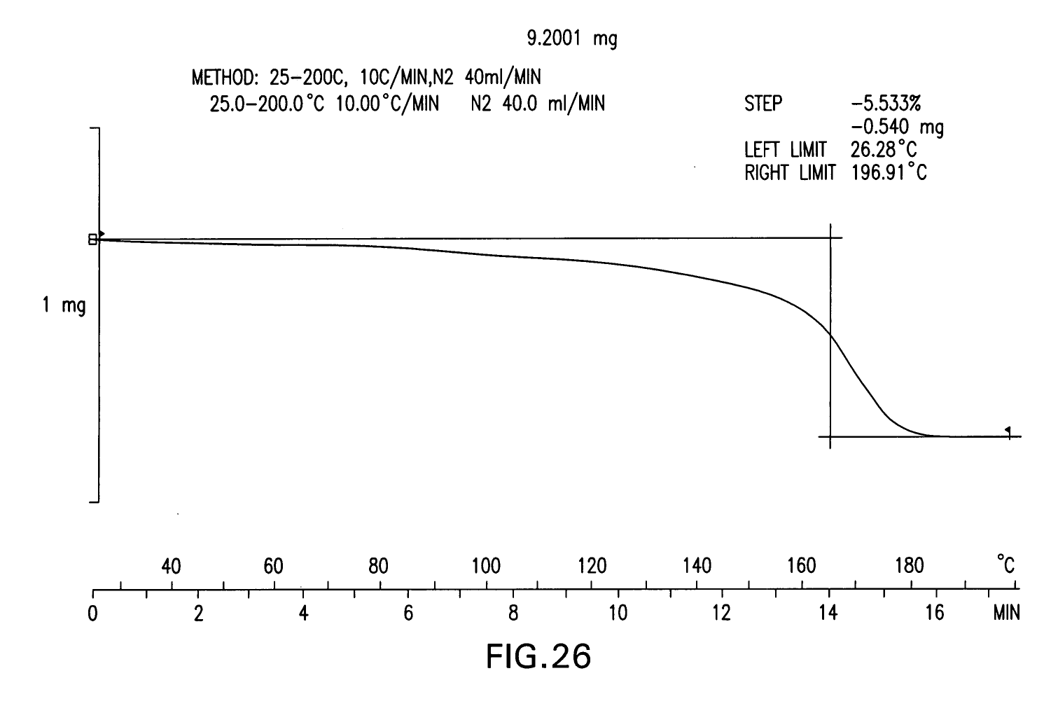

In another embodiment, the present invention provides a crystalline form of tadalafil Form VII, toluene solvate characterized by at least one of: an x-ray diffraction pattern with reflections at about 7.0°, 13.1°, 17.6°, 19.0°, 20.9°, 24.6° 2θ, or by two endotherms in DSC: a broad endotherm at about 170° C. and a melting endotherm at about 300° C. The crystalline form may be a toluene solvate. Tadalafil form VII may be further characterized by TGA, showing a weight loss of about 5-6% at a temperature of about 80° C. Tadalafil form VII may be further characterized by Karl-Fisher, showing water content of less than 1%. FIG. 7 depicts a representative x-ray diffraction pattern of crystalline tadalafil Form VII. FIGS. 17 and 26 depict representative DSC and TGA thermograms of crystalline tadalafil Form VII, respectively.

In yet another aspect, the present invention provides a method of preparing crystalline tadalafil Form VII including the steps of providing a slurry of toluene and tadalafil, wherein the tadalafil is selected from the group of crystalline forms consisting of crystalline tadalafil Form IV, crystalline tadalafil Form V, and crystalline tadalafil Form II until a precipitate is obtained; and isolating the precipitate. Preferably, the isolation is by filtration. Preferably, the isolated precipitate is dried at about 65° C.

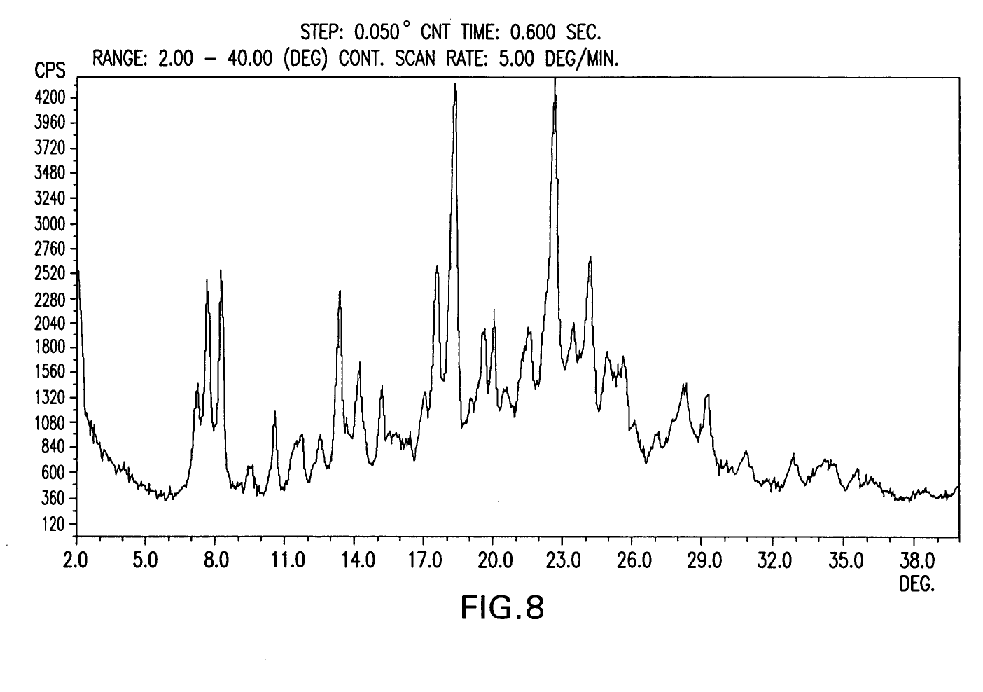

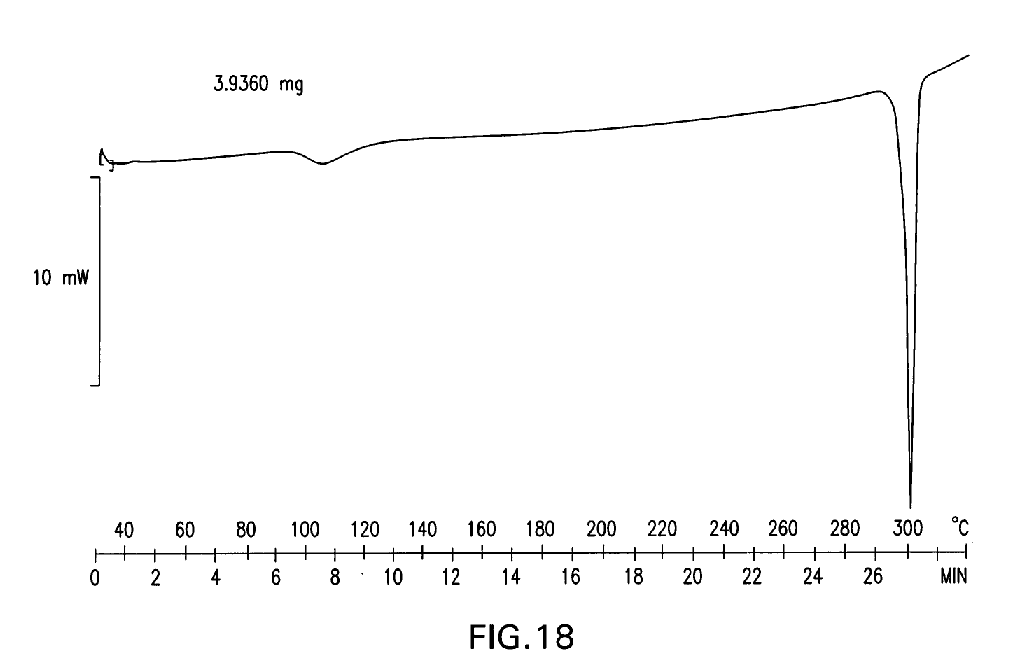

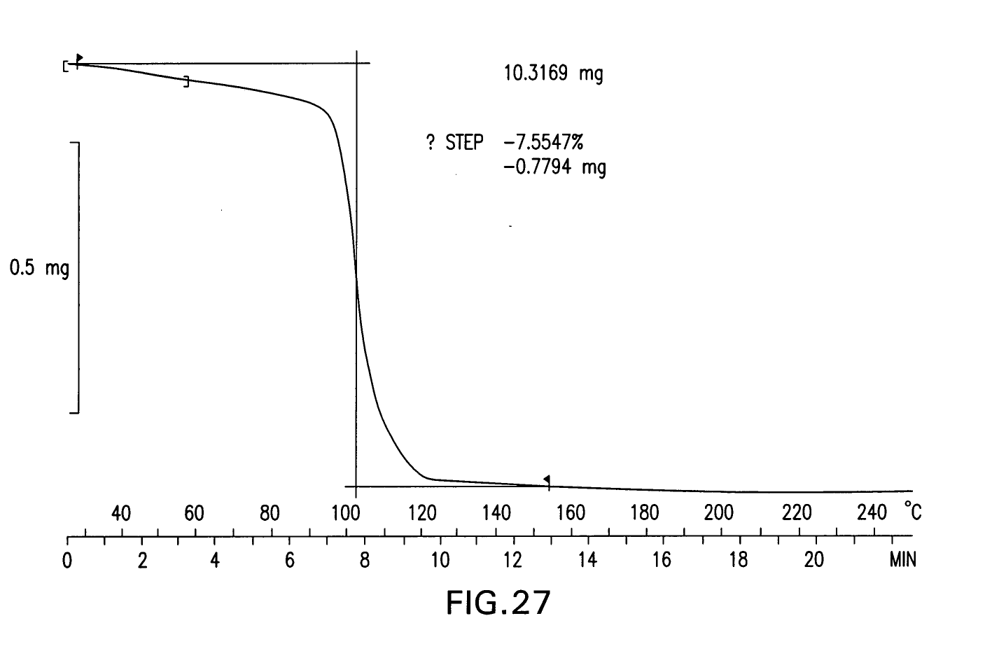

In another embodiment, the present invention provides crystalline tadalafil Form VIII, dichloromethane solvate, characterized by an x-ray diffraction pattern with reflections at about 7.2°, 7.6°, 8.2°, 13.3°, 17.6°, 18.2°, 22.6°±2° 2θ. The crystalline form may be dichloromethane solvate. The crystalline form may be further characterized by two endotherms at about 100° C. and at about 300° C. Tadalafil form VIII may be further characterized by TGA, showing a weight loss of about 7-9%. FIG. 8 depicts a representative x-ray diffraction pattern of crystalline tadalafil Form VIII. FIGS. 18 and 27 depict representative DSC and TGA thermograms, respectively, of crystalline tadalafil Form VIII.

In another embodiment, the present invention provides a process for preparing crystalline tadalafil Form VIII, dichloromethane solvate by heating. In this method, crystalline tadalafil form IV can either be heated to a temperature of between about 50° C. to about 70° C., preferably to about 65° C., preferably under vacuum, to obtain crystalline tadalafil Form VIII, dichloromethane solvate, or can be heated to about a temperature of between about 40° C. to about 70° C., preferably to about 60° C., preferably under atmospheric pressure, to obtain a mixture of crystalline tadalafil forms, wherein the forms are crystalline anhydrous tadalafil Form I, and crystalline tadalafil Form VIII, dichloromethane solvate.

In yet another embodiment, the present invention provides a method of preparing crystalline tadalafil Form I, by heating crystalline tadalafil Form IV at about a temperature of between about 40° C. to about 80° C., preferably to about 60° C., to obtain a mixture of crystalline tadalafil forms, wherein the crystalline forms are Form VIII and Form I. Preferably, heating is under atmospheric pressure.

Another embodiment of the invention encompasses crystalline forms II, III, IV, VI, VII and VIII of tadalafil substantially free of crystalline Form I.

Crystalline forms II, III, IV, VI, VII and VIII of tadalafil may be obtained with a particle size distribution, of particles having d(0.9), of about 200μ o about 600μ. The particles size distribution of Tadalafil crystalline forms of the present invention may vary by changing experimental parameters, such as cooling rate, and speed of agitation.

The particles size distribution of Form I may be of about 200μ to about 600μ, after milling.

X-ray diffraction data was obtained with a Scintag, variable goniometer, Cu-tube, solid state detector, using a round standard aluminum sample holder with round zero background. Scanning parameters: Range 2-40° 2θ: continuous scan at a rate of 3°/min.

DSC data was obtained with a DSC821^{e}, Mettler Toledo. The sample weight was about 3-5 mg, and the heating (scan) rate was about 10° C./min. The lid of the crucible had 3 holes in it.

TGA data was obtained using a Mettler TG50 using standard alumina pan. The sample weight was 7-15 mg, and the heating (scan) rate was about 10°/min.

A cold room thermostatted between 10° C. and 30° C. was used in several of the following examples. The temperature was changed according to the needs and objectives of the experiment.

### EXAMPLES

### Preparation of Tadalafil Crystal Form I

### Example 1

Tadalafil (5.01 g) was added to an Erlenmeyer flask with 2-methoxyethanol (104 ml) and heated in a water bath at about 83° C. until dissolved. The solution was cooled to room temperature, and after about 24 hours, was further cooled in a cold room about 24 hours. The precipitate was collected by filtration and dried in a vacuum oven for about 3 hours at about 65° C. Tadalafil form I was obtained. Loss on drying (“LOD”) by TGA was 0.6%.

### Example 2

Tadalafil (5.07 g) was added to an Erlenmeyer flask with absolute ethanol (950 ml) and heated in a water bath at about 83° C. until dissolved. The solution was cooled to room temperature, and after about 24 hours, was further cooled in a cold room for about 24 hours. The precipitate was collected by filtration and dried in a vacuum oven for about 3 hours at about 65° C. Tadalafil form I was obtained. LOD by TGA was 0.3%.

### Example 3

Tadalafil (5.07 g) was added to an Erlenmeyer flask with acetonitrile (250 ml) and heated in a water bath at about 83° C. until dissolved. The solution was cooled to room temperature, and after about 24 hours, was further cooled in a cold room for about 24 hours. The precipitate was collected by filtration and dried in a vacuum oven for about 3 hours at about 65° C. Tadalafil form I was obtained.

### Example 4

Tadalafil (5.14 g) was added to an Erlenmeyer flask with 1-propanol (1 L) and heated in a water bath at about 83° C. until dissolved. The solution was cooled to room temperature, and after about 24 hours, was further cooled in a cold room for about 24 hours. The precipitate was collected by filtration and dried in a vacuum oven for about 3 hours at about 65° C.

### Example 5

Tadalafil (4.18 g) was added to an Erlenmeyer flask with isopropanol (1 L) and heated in a water bath at about 83° C. until dissolved. The solution was cooled to room temperature, and after about 24 hours, was further cooled in a cold room for about 24 hours. The precipitate was collected by filtration and dried in a vacuum oven for about 3 hours at about 65° C.

### Example 6

Tadalafil (5.0 g) was stirred in ethyl acetate (950 ml) and heated to reflux temperature. The solution was left overnight to crystallize, and was further cooled in an ice bath for about 1 hour. The precipitate was then collected by filtration.

### Example 7

Tadalafil (5.0 g) was stirred in toluene (160 ml) and DMSO (4 ml) and heated to reflux temperature. The solution was left overnight to crystallize, and was further cooled in an ice bath for about 1 hour. The precipitate was then collected by filtration

### Example 8

Tadalafil (5.0 g) was stirred in n-butanol (300 ml) and heated to reflux temperature. The solution was left overnight to crystallize, and was further cooled in an ice bath for about 1 hour. The precipitate was then collected by filtration.

### Example 9

Tadalafil (5.0 g) was stirred in methanol (850 ml) and heated to reflux temperature. The solution was left overnight to crystallize, and was further cooled in an ice bath for about 1 hour. The precipitate was then collected by filtration.

Example 10

Tadalafil (5.0 g) was stirred in chloroform (225 ml) and heated to reflux temperature. The solution was left overnight to crystallize, and was further cooled in an ice bath for about 1 hour. The precipitate was then collected by filtration.

### Example 11

Tadalafil (5.0 g) was stirred in THF (150 ml) and heated to reflux temperature. The solution was left overnight to crystallize, and was further cooled in an ice bath for about 1 hour. The precipitate was then collected by filtration.

### Example 12

Tadalafil (5.0 g) was dissolved in chloroform (300 ml). Petroleum ether 40-60 (200 ml) was added to the solution, and the resulting precipitate was collected by filtration.

### Example 13

Tadalafil (5.0 g) was dissolved in methylene chloride (300 ml). Cyclohexane (200 ml) was added to the solution, and the resulting precipitate was collected by filtration.

### Example 14

Tadalafil (5.0 g) was dissolved in THF (20 ml). Petroleum ether 40-60 (500 ml) was added to the solution, followed by an addition of methanol (150 mls). The resulting precipitate was then collected by filtration.

### Example 15

Tadalafil (5.0 g) was dissolved in chloroform (300 ml). Toluene (800 ml) was then added to the solution, and the resulting precipitate was collected by filtration.

### Example 16

Tadalafil (5.0 g) was dissolved in chloroform (300 ml). Mixture of Xylenes (620 ml) were then added to the solution, and the resulting precipitate was collected by filtration.

### Example 17

Tadalafil (5.0 g) was dissolved in chloroform (300 ml). Benzene (700 ml) was then added to the solution, and the resulting precipitate was collected by filtration.

### Example 18

Tadalafil (5.0 g) was dissolved in acetone (400 ml) at 55° C. MTBE (400 ml) was added to the solution, and the resulting precipitate was collected by filtration.

### Example 19

Tadalafil (5.0 g) was dissolved in methylethyl ketone (400 ml) at 80° C. Petroleum ether 40-60 (400 ml) was added to the solution, and the resulting precipitate was collected by filtration. The sample was then dried at 65° C. overnight under atmospheric pressure to yield a mixture of Form I and solvated Form III.

### Example 20

Methylethyl ketone (750 ml) was added to a 1L Erlenmeyer flask and heated to 80° C. in a water bath. Tadalafil (12.4 g) was slowly added. An additional small amount of methylethyl ketone was added to insure total dissolution. The solution was removed from the heating bath. It crystallized, was stirred overnight, and filtered the following day. The sample was then dried at 65° C. overnight under atmospheric pressure to yield a mixture of Form I and solvated Form III.

### Example 21

Tadalafil (5.0 g) was dissolved in hot methylethyl ketone (400 ml). Petroleum ether 40-60 (400 ml) was added and the resulting precipitate was filtered. The sample was held at 50° C. for 7 days to yield Form I.

### Example 22

Tadalafil (5.0 g) was dissolved in hot methylethyl ketone (400 ml). Petroleum ether 40-60 (400 ml) was added to the solution, and the resulting precipitate was filtered. The sample was analyzed by XRD and by TGA and found to contain Tadalafil form II MethylEthyl Ketone solvate. The sample was held at room temperature at 100% humidity for 7 days to yield Form I.

### Example 23

Methylethyl ketone (750 ml) was added to a 1 L Erlenmeyer flask and heated in a water bath. Tadalafil (12.4 g) was slowly added. An additional small amount of methylethyl ketone was added to insure total dissolution. The solution was removed from the heating bath. It crystallized, was stirred overnight, and was filtered the following day. The sample was identified by XRD and by TGA analyses to contain form II Methyl Ethyl Ketone solvate. The sample was held at room temperature at 100% relative humidity for 7 days to yield Form I.

### Example 24

Tadalafil (5.09 g) was heated with acetone (326 ml) at 83° C. in a water bath until dissolution was complete. The solution was removed from the water bath and cooled to room temperature. After 24 hours of standing, the sample was placed in a cold room for 24 hours, filtered, and dried at 65° C. for 24 hours.

### Example 25

Tadalafil (5.09 g) was heated with acetone (326 ml) at 83° C. in a water bath until dissolution was complete. The solution was removed from the water bath and cooled to room temperature. After 24 hours of standing, the sample was placed in a cold room for 24 hours, and then filtered. The sample was held for 3 days at room temperature in 100% relative humidity to yield Form I.

### Preparation of Tadalafil Crystal Form II

### Example 26

Tadalafil (5.14 g) was added to an Erlenmeyer flask with methylethyl ketone (346 ml) and heated in a water bath at about 83° C. until dissolved. The solution was cooled to room temperature, and after about 24 hours, was further cooled in a cold room for about 24 hours. The resulting precipitate was collected by filtration. Water content by Karl Fischer (“KF”) was 0.46%. Tadalafil form II was obtained.

### Example 27

Tadalafil (5.09 g) was added to an Erlenmeyer flask with acetone (326 ml) and heated in a water bath at about 83° C. until dissolved. The solution was cooled to room temperature, and after about 24 hours, was further cooled in a cold room at about 10-30° C. for about 24 hours. The resulting precipitate was collected by filtration. Tadalafil form II was obtained.

### Example 28

Tadalafil (5.0 g) was dissolved in methylethyl ketone (400 ml) at 80° C. Petroleum ether (400 ml) was added to the solution, and the resulting precipitate was collected by filtration. Tadalafil form II was obtained.

### Example 29

Tadalafil (5.0 g) was dissolved in hot methylethyl ketone (400 ml). Cyclohexane (400 ml) was added to the solution, and the resulting precipitate was collected by filtration. Tadalafil form II was obtained.

### Example 30

Tadalafil (5.0 g) was dissolved in hot methylethyl ketone (400 ml). MTBE (400 ml) was added to the solution, and the resulting precipitate was collected by filtration. Tadalafil form II was obtained. Water content by KF was 0.11%.

### Preparation of Tadalafil Crystal Form III

### Example 31

Tadalafil prepared according to Example 26 was dried at about 65° C. under vacuum for about 3 hours, resulting in a mixture of Forms II and III.

### Example 32

Tadalafil prepared according to Example 27 was dried at about 65° C. under vacuum for about 3 hours, resulting in a mixture of Forms II and III.

### Example 33

Tadalafil methylethyl ketone solvate Form II was heated at atmospheric pressure at about 65° C. for about 2 hours, resulting in a mixture of Forms I and III.

### Example 34

Tadalafil acetone solvate Form II was heated at 65° C. under vacuum for about 3 hours, resulting in Tadalafil Form III. Water content by KF was 0.4%.

### Preparation of Tadalafil Crystal Form IV

### Example 35

Tadalafil (5.0 g) was stirred in methylene chloride (450 ml) and heated to reflux temperature. It was left overnight to crystallize, and then further cooled in an ice bath for about 1 hour to complete precipitation. The precipitate was then filtered. Tadalafil form IV was obtained. Water content by KF was 0.21%.

### Example 36

Tadalafil (5.0 g) was dissolved in methylene chloride (700 ml). 40-60 petroleum ether (300 ml) was added to the solution, and the resulting precipitate was collected by filtration. Tadalafil form IV was obtained. Water content by KF was 0.38%.

### Preparation of Tadalafil Crystal Form V

### Example 37

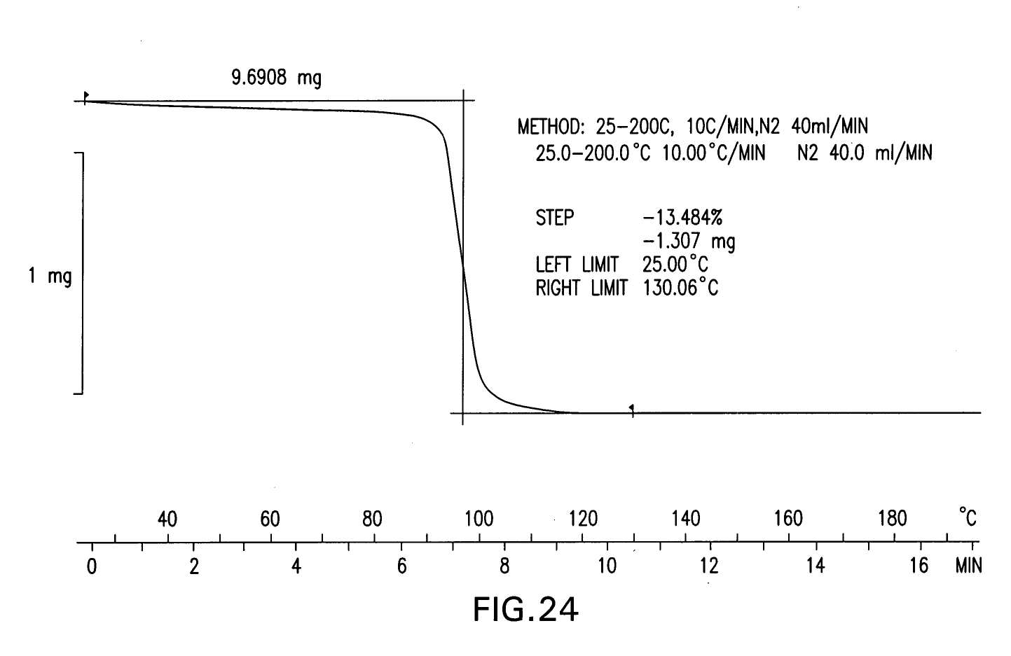

Tadalafil (5.0 g) was stirred in acetic acid (50 ml) and heated to reflux temperature. It was left overnight to crystallize, and then further cooled in an ice bath for 1 hour. The resulting precipitate was then collected by filtration. Water content by KF was 0.20%. Tadalafil form V was obtained. See TGA thermogram in FIG. 24.

### Preparation of Tadalafil Crystal Form VI

### Example 38

Tadalafil Form IV (2.0 g) was slurried overnight in methanol (15 ml) and filtered the following day. The sample was dried at 65° C. under vacuum for 3 h. Tadalafil form VI was obtained. Water content by KF was less than about 1%.

### Preparation of Tadalafil Crystal Form VII

### Example 39

Tadalafil Form IV (2.0 g) was slurried overnight in toluene (20 ml) and filtered the following day. The sample was dried at 65° C. under vacuum for 3 h. Tadalafil form VII was obtained.

### Example 40

Tadalafil Form V (2.0 g) was slurried in toluene (20 ml) overnight, and filtered the following day. The sample was dried at 65° C. under vacuum for 3 h. Tadalafil form VII was obtained.

### Example 41

Tadalafil Form II (2.0 g) was slurried overnight in toluene (20 ml) and filtered the following day. The sample was dried at 65° C. under vacuum for 3 h. Tadalafil form VII was obtained.

### Preparation of Tadalafil Crystal Form VIII

### Example 42

Tadalafil dichloromethane solvate Form IV (2.0 g) was heated at 65° C. under vacuum. Tadalafil form VIII was obtained.

### Example 43

Tadalafil dichloromethane solvate Form IV (0.5 g) was heated at 60° C. under atmospheric pressure to obtain a mixture of crystalline tadalafil forms Form I and Form VIII.

What is claimed is:

1. A process for preparing crystalline tadalafil form I comprising crystallizing it from a solvent selected from the group consisting of: 2-methoxyethanol, absolute ethanol, acetonitrile, 1-propanol, isopropanol, ethyl acetate, toluene and dimethyl sulfoxide (“DMSO”), n-butanol, chloroform, tetrahydrofuran (“THF”) and mixtures thereof.

2. A process for preparing the crystalline tadalafil form I, comprising the steps of:
a) dissolving tadalafil in a solvent selected from the group consisting of 2-methoxyethanol, absolute ethanol, acetonitrile, 1-propanol, isopropanol, and mixtures thereof, at a temperature of at least about 60° C. to about 120° C. to obtain a solution;
b) cooling the tadalafil solution of step a) until a precipitate is obtained; and
c) isolating the precipitate of step b).

3. The process of claim 1, wherein the solution in step b) is cooled to a temperature below about 30° C. and above about 10° C.

4. The process of claim 1, wherein the solution in step b) is cooled to about room temperature.

5. A process for preparing the crystalline tadalafil form I, comprising the steps of:
a) dissolving tadalafil in a solvent selected from the group consisting of ethyl acetate, toluene containing about DMSO, n-butanol, methanol, chloroform, THF and mixtures thereof to obtain a solution;
b) cooling the tadalafil solution of step a) until a precipitate is obtained; and
c) isolating the precipitate of step b) to obtain crystalline tadalafil.

6. The process of claim 5, wherein the tadalafil in step a) is dissolved at reflux temperature.

7. The process of claim 5, wherein the solution in step b) is cooled to a temperature below about room temperature and above about 0° C.

8. A process for preparing the crystalline tadalafil form I, comprising the steps of:
a) dissolving tadalafil in a solvent selected from the group consisting of chloroform, methylene chloride, THF, and acetone to obtain a solution;
b) combining the solution of step a) with an anti-solvent selected from the group consisting of petroleum ether, cyclohexane, toluene, xylenes, benzene, hexane, heptane, octane, and MTBE, until a precipitate is obtained; and
c) isolating the precipitate of step b) to obtain crystalline tadalafil.

9. The process of claim 8 wherein the solvent of step a) is chloroform and the anti-solvent of step b) is selected from the group consisting of: petroleum ether, wherein the petroleum ether is about 40% final volume, toluene, wherein the toluene is about 73% final volume, the xylenes, wherein the xylenes are about 70% final volume, and benzene, wherein benzene is about 70% final volume.

10. The process of claim 8 wherein the solvent of step a) is THF and the anti-solvent comprises first and second anti-solvents, wherein the first anti-solvent is petroleum ether, wherein the petroleum ether is about 96% volume after combination with the tadalafil solution, and wherein the second anti-solvent is methanol, wherein methanol is about 23% final volume.

11. The process of claim 8 wherein the solvent of step a) is methylene chloride and the anti-solvent of step b) is cyclohexane, wherein the cyclohexane is about 40% final volume.

12. The process of claim 8 wherein the solvent of step a) is acetone and the anti-solvent of step b) is MTBE, wherein the MTBE is about 50% final volume.

13. A process for preparing the crystalline tadalafil form I, comprising the steps of:
a) dissolving tadalafil in THF to obtain a solution;
b) combining the solution of step a) with an anti solvent selected from the group consisting of: petroleum ether, heptane and hexane;
c) adding an anti-solvent that is methanol until a precipitate is obtained; and
d) isolating the precipitate of step c) to obtain crystalline tadalafil.

14. A process for preparing the crystalline tadalafil form I, comprising the steps of:
a) dissolving tadalafil in an aliphatic ketone selected from the group consisting of methylethyl ketone, isobutyl ketone or acetone to obtain a solution;
b) cooling the solution until a precipitate is obtained; and
c) drying the precipitate of step c) at a temperature of about 45° C. to about 90° C. to obtain crystalline tadalafil.

15. The process of claim 14, wherein the solution is cooled to room temperature.

16. The process of claim 15, wherein the solution is further cooled to a temperature of less than about 10° C.

17. The process of claim 14, wherein the precipitate in step c) is dried to about 65° C.

18. The process of claim 14, wherein the precipitate in step c) is dried under atmospheric pressure.

19. A process for preparing the crystalline tadalafil form I, comprising the steps of:
a) dissolving tadalafil in an aliphatic ketone selected from the group consisting of methylethyl ketone and acetone to obtain a solution;
b) cooling the solution until a precipitate is obtained;
c) isolation the precipitate; and
d) exposing the precipitate to high humidity to obtain crystalline tadalafil.

20. The process of claim 19, wherein the solution in step b) is cooled to about room temperature.

21. The process of claim 20, wherein the solution is further cooled to a temperature of less than about 10° C.

22. A crystalline form of tadalafil (Form II) characterized by x-ray reflections at about 7.6°, 14.0°, 15.2°, 18.0°, and 22.8°±2° 2θ.

23. The crystalline tadalafil of claim 22 having an x-ray diffraction diagram substantially as depicted in FIG. 2.

24. The crystalline form of claim 22, characterized by two endotherms in DSC at about 105-115° C. and at about 300° C.

25. The crystalline form of claim 22, characterized by TGA, showing a weight loss of about 10-15% at a temperature of below about 120° C.

26. The crystalline tadalafil of claim 22, wherein the crystalline tadalafil is a ketone solvate.

27. The crystalline tadalafil of claim 26 wherein the ketone solvate is methylethyl ketone solvate.

28. The crystalline tadalafil of claim 26 wherein the ketone solvate is acetone solvate.

29. A process for preparing the crystalline form of tadalafil of claim 22 comprising the steps of:
a) providing a solution of tadalafil in a solvent selected from the group consisting of methylethyl ketone and acetone, at a temperature of about 45° C. to about 83° C.;
b) cooling the solution of step a) until a precipitate is obtained; and
c) isolating the precipitate of step b) to obtain the crystalline tadalafil of claim 22.

30. The process of claim 29, wherein tadalafil in step a) is provided at about 83° C.

31. The process of claim 29, wherein the solution in step b) is cooled to a temperature below about 0° C. and above about 25° C.

32. The process of claim 31, wherein the solution in step b) is cooled to about 10° C.

33. A process for preparing the crystalline form of tadalafil of claim 22 comprising the steps of:
a) dissolving tadalafil in methylethyl ketone to obtain a solution;
b) combining the solution of step a) with an anti-solvent selected from the group consisting of petroleum ether, cyclohexane, and MTBE, until a precipitate is obtained; and
c) isolating the precipitate of step b) to obtain crystalline tadalafil.

34. A process for preparing crystalline tadalafil Form I comprising the steps of drying crystalline Tadalafil form II ketone solvate at a temperature of about 40° C. to about 90° C.

35. The process of claim 34, wherein the drying is for at least 2 days.

36. The process of claim 34, wherein the drying is under atmospheric pressure.

37. The process of claim 34, wherein the drying is at a temperature of about 50° C.

38. A process for preparing crystalline tadalafil Form I comprising the steps of exposing crystalline tadalafil selected from the group consisting of crystalline tadalafil Form II, methylethyl ketone solvate and crystalline tadalafil Form II, acetone solvate to high humidity.

39. A crystalline form of tadalafil (Form III) characterized by x-ray reflections at about 8.3°, 13.5°, 7.7°, and 18.4°±2° 2θ.

40. The crystalline form of tadalafil of claim 39 having an x-ray diffraction diagram substantially as depicted in FIG. 3.

41. The crystalline form of tadalafil of claim 39, characterized by two endotherms in DSC at about 80-90° C. and at about 300° C.

42. The crystalline form of tadalafil of claim 39, characterized by TGA, showing a weight loss of about 4-5% at a temperature of about 80° C.

43. The crystalline tadalafil of claim 39, wherein the crystalline tadalafil is a ketone solvate.

44. The crystalline tadalafil of claim 43, wherein the ketone solvate is methylethyl ketone solvate.

45. The crystalline tadalafil of claim 43, wherein the ketone solvate is acetone solvate.

46. A process for preparing the crystalline form of tadalafil of claim 39 comprising one of the following:
a) drying crystalline tadalafil Form II, at a temperature of about 50° C. to about 80° C. under vacuum for about 0.5 to about 6 hours until obtaining a mixture of crystalline tadalafil Form II and Form III; or
b) drying the tadalafil methylethyl ketone solvate Form II, at about 45° C. to about 70° C. under vacuum for about 0.5 to about 5 hours to obtain a mixture of crystalline tadalafil Form II and Form III; or
c) drying the tadalafil acetone solvate Form II, at about 45° C. to about 70° C. under vacuum for about 0.5 to about 5 hours to obtain crystalline tadalafil, designated Form III.

47. The process of claim 46, wherein the drying in a), b) or c) is at a temperature of about 65° C.

48. The process of claim 46, wherein the drying in a), b) or c) is under vacuum.

49. A process for preparing the crystalline tadalafil Form I comprising exposing crystalline tadalafil selected from the group consisting of crystalline tadalafil Form III, methylethyl ketone solvate and crystalline tadalafil Form III, acetone solvate to high humidity.

50. A process for preparing a mixture of crystalline tadalafil Form I and crystalline tadalafil form III comprising the steps of drying the crystalline tadalafil selected from the group consisting of crystalline tadalafil Form II, methylethyl ketone solvate and crystalline tadalafil Form II, acetone solvate, at a temperature of between about 50° C. to about 75° C.

51. The process of claim 50, wherein the drying under atmospheric pressure.

52. The process of claim 50, wherein the drying is to a temperature of about 65° C.

53. A crystalline form of tadalafil (Form IV) characterized by x-ray reflections at about 7.6°, 10.6°, 15.2°, 18.4°, and 22.7°±2° 2θ.

54. The crystalline form of tadalafil of claim 53 having an x-ray diffraction diagram substantially as depicted in FIG. 4.

55. The crystalline form of tadalafil of claim 53, characterized by two endotherms in DSC at about 110-115° C. and at about 300° C.

56. The crystalline form of tadalafil of claim 53, characterized by TGA, showing a weight loss of about 11-16%.

57. A process for preparing the crystalline form of tadalafil of claim 53 comprising the steps of:
a) dissolving tadalafil in methylene chloride to obtain a solution;
b) cooling the solution of step a), until a precipitate is obtained; and
c) isolating the precipitate of step c) to obtain the crystalline tadalafil.

58. The process of claim 57, wherein the dissolving in step a) is at about reflux temperature.

59. The process of claim 57, wherein the solution in step b) is cooled to a temperature of between about 0° C. to about room temperature.

60. A process for preparing the crystalline form of tadalafil of claim 53 comprising the steps of:
a) dissolving tadalafil in methylene chloride to obtain a solution;
b) combining the solution of step a) with petroleum ether; and
c) isolating the precipitate of step b) to obtain crystalline tadalafil.

61. The process of claim 60, wherein the dissolving in step a) is at about reflux temperature.

62. A process for preparing the crystalline form V of tadalafil comprising the steps of:
a) dissolving tadalafil in acetic acid to obtain a solution;
b) cooling the solution of step a) to obtain a precipitate; and
c) isolating the precipitate of step b) to obtain the crystalline tadalafil.

63. The process of claim 62, wherein the dissolving in step a) is at about reflux temperature.

64. The process of claim 62, wherein the cooling in step b) is to a temperature of between about room temperature to about 0° C.

65. A crystalline form of tadalafil (Form VI) characterized by at least one of:
a) x-ray reflections at about 7.1°, 9.3°, 11.4°, 13.5°, 17.8°, 19.2°, 21.2° 2θ, or
b) an exotherm in DSC at about 200° C. and a melting endotherm at about 300° C.

66. The crystalline form of tadalafil of claim 65, having an x-ray diffraction diagram substantially as depicted in FIG. 6.

67. The crystalline form of tadalafil of claim 65, having a DSC thermogram substantially as depicted in FIG. 16.

68. The crystalline form of tadalafil of claim 65, characterized by TGA, showing a weight loss of less than 1%.

69. A process for preparing the crystalline form of tadalafil of claim 65 comprising the steps of:
a) providing a slurry of methanol and crystalline tadalafil Form IV; and
b) isolating the tadalafil from step a) to obtain crystalline tadalafil.

70. The process of claim 69, wherein the isolated form is further dried at a temperature of about 40° C. to about 70° C. under vacuum.

71. The process of claim 70, wherein the isolated crystalline tadalafil is dried at a temperature of about 65° C.

72. The process of claim 69, wherein the drying is for about 3 hours.

73. A crystalline form of tadalafil (Form VI) characterized by at least one of the following:
a) x-ray reflections at about 7.0°, 13.1°, 17.6°, 19.0°, 20.9°, 24.6° 2θ; or
b) two endotherms in DSC at about 170° C. and about 300° C.

74. The crystalline form of tadalafil of claim 73, having an x-ray diffraction diagram substantially as depicted in FIG. 7.

75. The crystalline form of tadalafil of claim 73, having a DSC thermogram substantially as depicted in FIG. 17.

76. The crystalline form of tadalafil of claim 73, wherein the crystalline form is a toluene solvate.

77. A process for preparing the crystalline form of tadalafil of claim 73 comprising the steps of:
a) slurrying tadalafil in toluene, wherein the tadalafil is selected from a group of crystalline forms consisting of: Form IV, Form V, and Form II, until a precipitate is obtained; and
b) isolating the precipitate of step a) to obtain crystalline tadalafil.

78. The process of claim 77, wherein the isolated form is further dried at about 65° C. under vacuum.

79. The process of claim 78, wherein the drying is for about 3 hours.

80. A crystalline form of tadalafil (Form VIII) characterized by x-ray reflections at about 7.2°, 7.6°, 8.2°, 13.3°, 17.6°, 18.2°, 22.6°±2° 2θ.

81. The crystalline form of tadalafil of claim 80 having an x-ray diffraction diagram substantially as depicted in FIG. 8.

82. The crystalline form of claim 80, characterized by two endotherms in DSC at about 100° C. and about 300° C.

83. The crystalline form of tadalafil of claim 80 wherein the crystalline form is a dichloromethane solvate.

84. A process for preparing the crystalline form of tadalafil of claim 80 comprising one of the following steps:
a) heating crystalline tadalafil Form IV to a temperature of about 50° C. to about 70° C.; or
b) heating crystalline tadalafil Form IV to a temperature of about 40° C. to about 70° C. to obtain a mixture of crystalline tadalafil Form I and Form VIII.

85. The process of claim 84, wherein the crystalline tadalafil Form IV in a) is heated to a temperature of about 65° C.

86. The process of claim 84, wherein the heating in a) is under vacuum.

87. The process of claim 84, wherein the crystalline tadalafil Form IV in b) is heated to a temperature of about 60° C.

88. The process of claim 84, wherein the heating in b) is under atmospheric pressure.

89. A process for preparing crystalline anhydrous tadalafil Form I, wherein crystalline tadalafil Form IV is heated to a temperature of about 40° C. to about 80° C., to obtain a mixture of crystalline tadalafil forms, wherein the crystalline forms are denominated Form I and Form VIII.

90. The process of claim 89, wherein the heating is to a temperature of about 60° C.

91. The process of claim 89, wherein the heating is under atmospheric pressure.
